(19) United States
(12) Patent Application Publication
(10) Pub. No.: US 2019/0032072 A1
(43) Pub. Date: Jan. 31, 2019

(54) METHOD FOR MODIFYING LATERAL BUDDING

(71) Applicant: BRITISH AMERICAN TOBACCO (INVESTMENTS) LIMITED, London (GB)

(72) Inventors: Gwendoline Leach, London (GB); Juan Pablo Sanchez Tamburrino, Cambridge (GB); Matthew Edward Humphry, London (GB); Anne Deslattes Mays, Wageningen (NL); Jesse David Munkvold, Wageningen (NL)

(21) Appl. No.: 16/070,193

(22) PCT Filed: Jan. 12, 2017
(86) PCT No.: PCT/GB2017/050069

§ 371 Date: Jul. 13, 2018

(30) Foreign Application Priority Data
Jan. 15, 2016 (GB) 1600751.0
Jan. 20, 2016 (GB) 1601040.7

(51) Int. Cl. C12N 15/82

(52) U.S. Cl. CPC C12N 15/8261; C12N 15/8218

(57) ABSTRACT

The present invention relates to a method for modifying lateral budding in a plant comprising modifying the expression or function of a protein comprising the sequence shown as SEQ ID NO: 3, 4 or 5 or a sequence which has at least 70% sequence identity thereto. The invention further relates to plants, plant propagation material, harvested leaf and processed leaf obtainable by such methods.

### FIELD OF THE INVENTION

The present invention relates to a method for modifying lateral budding in a plant and to a cell, plant, plant propagation material, harvested leaf, processed leaf or product derived therefrom.

### BACKGROUND

The control of plant morphology is of major importance in the commercial production of plants for agricultural or horticultural purposes, to enhance productivity and yield, to improve the efficiency of husbandry and harvest, and to achieve aesthetic desirability.

Morphological changes often occur as a result of environmental impact on the plant, including physical damage, herbivore predation, pathogen infection, cold, heat, and drought. They can often be brought about by human intervention, either physically (pruning, bending, typing, staking, or excising particular organs or structures) or chemically (application of agrochemicals and plant growth structures).

A particular application of controlling morphological changes to modify plant morphology would be in the modulation, preferably prevention or delay, of lateral shoot outgrowths from leaf axillary meristems. Outgrowth of lateral shoots most commonly arises when the dominance of the apical shoot is removed; for example when the apical shoot is damaged or removed, either accidentally through physical damage or predation by herbivores, or as part of agricultural practice e.g. topping. Other changes which modify, for example the production, transport, detection or metabolism of endogenous plant growth substances may also cause outgrowth from axillary meristems. Lateral shoots, or “suckers”, may be undesirable for purely aesthetic reasons, may produce a plant with unusable morphology, or may have a detrimental metabolic effect on the plant as a whole by acting as an additional source or sink for various metabolites or plant growth substances.

One example where lateral bud outgrowth occurs is in the commercial cultivation of plants of the Solanaceae family. For example, during the cultivation of tobacco plants, the apical shoot comprising the inflorescence and uppermost leaves is removed at a specific time during the growth of the plant, in a process named “topping”, to stimulate growth and development of the remaining leaves, to enhance root growth, and to encourage the redistribution of metabolites and secondary compounds to the plant leaves. A drawback to the topping process is that it also stimulates the outgrowth of lateral shoots which thereby offsets the desired redistribution of metabolites. This effect is commonly overcome by the physical removal of the lateral shoots, which is highly labour intensive, or by the application of chemical shoot suppressants such as maleic hydrazide, which is both costly in terms of the materials and may result in the retention of chemical residues on the harvested plant.

During the cultivation of tomato plants, suckers are commonly pruned in order to improve the production and health of the plant. However, pruning of suckers may cause unnecessary damage to the plant and may make the plant susceptible to disease.

In addition, there are circumstances in which it is desirable to increase lateral budding in plants, for example in certain field crops.

In addition, there are circumstances in which it is desirable to increase lateral budding in plants, for example in certain field crops.

A system which modifies, preferably reduces, such “suckering” by specifically targeting lateral bud outgrowth, would therefore provide a great benefit to the commercial cultivation of plants.

### SUMMARY OF THE INVENTION

According to a first aspect the present invention provides a method for modifying lateral budding in a plant comprising modifying the expression or function of a protein comprising the sequence shown as SEQ ID NO: 3, 4 or 5 or a sequence which has at least 70% sequence identity thereto.

In one embodiment the present invention provides a method for reducing and/or delaying lateral budding by reducing or preventing the expression or function of said protein.

In one embodiment the present invention provides a method for increasing and/or expediting lateral budding by increasing the expression or function of said protein.

In another aspect the present invention provides a plant cell obtainable (e.g. obtained) by a method according to the first aspect of the present invention.

In a further aspect the present invention provides a plant
i) obtainable by a method of the invention;
ii) comprising a modified nucleic acid sequence of the present invention;
iii) comprising a cell of the present invention.

In another aspect the present invention provides a plant propagation material (e.g. a plant seed) obtainable from a plant of the present invention.

In a further aspect the present invention provides a harvested leaf of a plant of the present invention or obtainable from a plant propagated from a propagation material of the present invention or obtainable from a plant obtainable by a method of the present invention.

In another aspect the present invention provides a processed leaf (preferably a non-viable processed leaf):
a. comprising a plant cell of the present invention;
b. obtainable from a plant obtainable from a method of the present invention;
c. obtainable from processing a plant of the present invention;
d. obtainable from a plant propagated from a plant propagation material of the present invention;
e. obtainable by processing a harvested leaf of the present invention.

In another embodiment the present invention provides a tobacco product:
a. prepared from a tobacco plant of the present invention or a part thereof;
b. prepared from a tobacco plant or a part thereof (preferably the leaves harvested from the plant) obtained or obtainable by the method of the present invention;
c. prepared from a tobacco plant (preferably the leaves) propagated from a plant propagation material of the present invention;
d. prepared from a harvested tobacco leaf of the present invention;
e. prepared from a processed tobacco leaf of the present invention;
f. prepared from or comprising a tobacco plant extract obtained from a tobacco plant of the present invention.

In a further aspect the present invention provides a plant extract of a plant according to the present invention or of a portion of said plant.

In a further aspect the present invention provides the use of a plant of the invention for breeding a plant.

In another aspect the present invention provides the use of a plant according to the present invention to grow a crop.

In another aspect the present invention provides the use of a plant according to the present invention to produce a leaf (e.g. a processed (preferably cured) leaf).

### BRIEF DESCRIPTION OF THE DRAWINGS

Embodiments of the invention will now be described, by way of example only, with reference to accompanying drawings, in which:

FIG. 1 shows lateral budding levels at 24 hours time intervals in control K326 plants and mutant TFA1280 plants as determined using digital phenotyping.

FIG. 2 shows lateral budding levels 14 days after topping in control K326 plants and mutant TFA1280 plants as determined using digital phenotyping.

FIG. 3 shows lateral budding levels in control K326 plants and mutant TFA1280 plants as determined by weight of lateral bud biomass.

FIG. 4 shows an example output image of the image analysis algorithm to generate pixel counts to determine sucker growth.

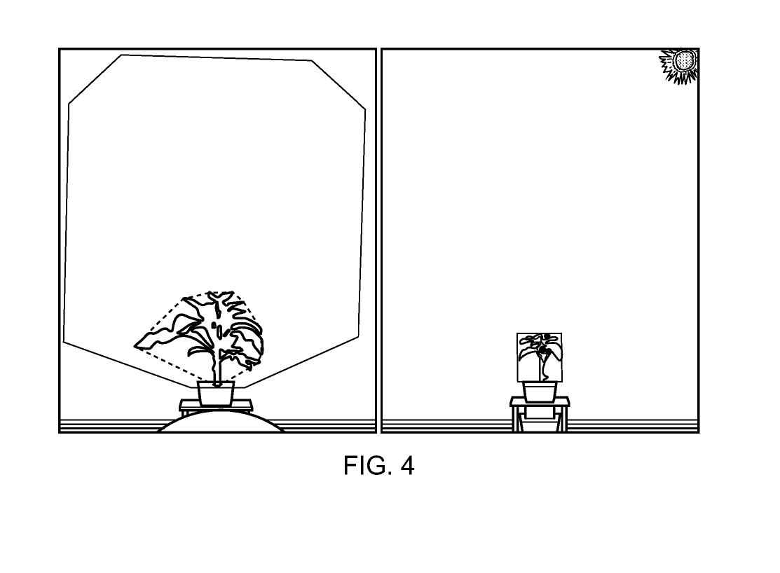

### DETAILED DESCRIPTION

For the first time the present inventors have surprisingly shown that lateral budding in a plant can be modified by modifying the expression or function of a protein comprising the amino acid sequence shown as SEQ ID NO: 3, 4 or 5 or an amino acid sequence which has at least 70% sequence identity thereto.

Lateral Budding

Lateral budding (suckering) refers to lateral shoot outgrowths from leaf axillary meristems. Outgrowth of lateral shoots most commonly arises when the dominance of the apical shoot is removed; for example when the apical shoot is damaged or removed, either accidentally through physical damage or predation by herbivores, or as part of agricultural practice e.g. topping. Other changes which modify, for example the production, transport, detection or metabolism of endogenous plant growth substances may also cause outgrowth from axillary meristems.

“Modifying lateral budding” is used herein to refer to altering the level or amount of lateral budding and/or lateral shoot growth in a plant. In particular, “modifying lateral budding” may refer to reducing/decreasing and/or delaying lateral budding and/or lateral shoot growth in a plant; or increasing or expediting lateral budding and/or lateral shoot growth in a plant.

In one embodiment “modifying lateral budding” may refer to reducing/decreasing and/or delaying lateral budding and/or lateral shoot growth in a plant.

In one embodiment “modifying lateral budding” may refer to reducing/decreasing lateral budding and/or lateral shoot growth in a plant.

In one embodiment lateral budding is reduced and/or delayed by carrying out a method of the invention to reduce or prevent the expression or function of a protein comprising the sequence shown as SEQ ID NO: 3, 4, 5 or an amino acid sequence which has at least 70% sequence identity thereto.

A reduction and/or delay in lateral budding in a plant, for example a tobacco plant, is a highly advantageous technical effect.

The terms “reducing lateral budding” or “reduction of lateral budding” are used herein to mean that the amount and/or level of lateral budding in a plant is lower in relation to a comparable plant. For example, a comparable plant would be a plant which had not been modified according to the present invention, but in which all other relevant features were the same (e.g. plant species, growing conditions etc).

“Reducing lateral budding” may refer to a fewer number of lateral buds and/or lateral shoots; a lower biomass of lateral buds and/or lateral shoots; and/or a lower growth rate of lateral buds and/or lateral shoots in relation to a comparable plant.

The term “delaying lateral budding” used herein to mean that lateral budding in a plant occurs later in a modified plant in accordance with the present invention compared with a comparable (control) plant. For example, a comparable (control) plant would be a plant which had not been modified according to the present invention, but in which all other relevant features were the same (e.g. plant species, growing conditions etc). The length of the delay may be dependent upon the plant species. However in some species, such as tobacco for instance the delay may be more than 2 weeks, preferably more than 4 weeks, preferable more than 6 weeks compared with a comparable plant which has not been modified according to the present invention.

In one embodiment carrying out a method of the invention results in a reduction of and/or delay in lateral budding when compared to a plant which has not been modified to reduce or prevent the expression or function of a protein comprising the sequence shown as SEQ ID NO: 3, 4 or 5 or an amino acid sequence which has at least 70% sequence identity thereto.

Any method known in the art for determining the amount and/or level of lateral budding may be used in the context of the present invention. For example, methods such as those detailed in the Examples described herein may be used. In particular, digital phenotyping of lateral bud growth or the weight of lateral bud biomass may be determined.

In one embodiment the amount and/or level of lateral budding may be reduced by at least about 1%, at least about 3%, at least about 5%, at least about 10%, at least about 20%, at least about 30%, at least about 40%, at least about 50%, at least about 60%, at least about 70%, at least about 80%, at least about 90%, at least about 95%, at least about 99% or 100% in relation to a comparable plant which has not been modified according to the present invention. In some embodiments the amount and/or level of lateral budding may be reduced by between about 5% and about 95%, by between about 10% and about 90%, by between 20% and about 80%, by between 30% and about 70%, or by between about 40% and 60% in relation to a comparable plant which has not been modified according to the present invention.

In one embodiment lateral budding is increased and/or expedited by carrying out a method of the invention to increase the expression or function of a protein comprising the sequence shown as SEQ ID NO: 3, 4 or 5 or an amino acid sequence which has at least 70% sequence identity thereto.

The term “increased lateral budding” is used herein to mean that the amount and/or level of lateral budding in a plant is greater in relation to a comparable plant. For example, a comparable plant would be a plant which had not been modified according to the present invention, but in which all other relevant features were the same (e.g. plant species, growing conditions etc).

“Increased lateral budding” may refer to a greater number of lateral buds and/or lateral shoots; an increased biomass of lateral buds and/or lateral shoots; and/or an increased growth rate of lateral buds and/or lateral shoots in relation to a comparable plant.

The term “expedited lateral budding” as used herein means that lateral budding in a plant occurs earlier in a modified plant in accordance with the present invention compared with a comparable (control) plant. For example, a comparable (control) plant would be a plant which had not been modified according to the present invention, but in which all other relevant features were the same (e.g. plant species, growing conditions etc). The exact timing of the lateral budding may be dependent upon the plant species. However in some species the lateral budding may be expedited my more than 2 weeks, preferably more than 4 weeks, preferable more than 6 weeks compared with a comparable plant which has not been modified according to the present invention.

In one embodiment carrying out a method of the invention results in an increase of and/or expedited lateral budding when compared to a plant which has not been modified to increase the expression or function of a protein comprising the sequence shown as SEQ ID NO: 3, 4 or 5 or an amino acid sequence which has at least 70% sequence identity thereto.

In one embodiment the amount and/or level of lateral budding may be increased by at least about 1%, at least about 3%, at least about 5%, at least about 10%, at least about 20%, at least about 30%, at least about 40%, at least about 50%, at least about 60%, at least about 70%, at least about 80%, at least about 90%, at least about 95%, at least about 99% or 100% in relation to a comparable plant which has not been modified according to the present invention. In some embodiments the amount and/or level of lateral budding may increased by between about 5% and about 95%, by between about 10% and about 90%, by between 20% and about 80%, by between 30% and about 70%, or by between about 40% and 60% in relation to a comparable plant which has not been modified according to the present invention.

Protein

As used herein, the term “protein” is synonymous with the term “polypeptide”. In some instances, the term “protein” is synonymous with the term “peptide”.

The terms “to reduce or prevent the expression or function of a protein” or “reduction or prevention of expression or function of a protein” are used herein to mean that the amount/level or activity of a protein comprising the amino acid sequence shown as SEQ ID NO: 3, 4 or 5 or an amino acid sequence which has at least 70% sequence identity thereto in the product, method or use of the invention is lower in relation to a comparable product, method or use. For example, a comparable product would be derived from a plant which had not been modified according to the present invention, but in which all other relevant features were the same (e.g. plant species, growing conditions, method of processing, etc).

The expression or function of a protein comprising the amino acid sequence shown as SEQ ID NO: 3, 4 or 5 or an amino acid sequence which has at least 70% sequence identity thereto may be reduced in a plant leaf, harvested plant leaf, processed plant leaf, plant product or combinations thereof obtainable or obtained from a plant of the invention when compared with a leaf, harvested plant leaf, processed plant leaf, plant product or combinations thereof obtainable or obtained from a comparable plant which has not been modified to reduce or prevent the expression of a protein comprising the amino acid sequence shown as SEQ ID NO: 3, 4 or 5 or an amino acid sequence which has at least 70% sequence identity thereto.

In one embodiment the expression or function of a protein comprising the amino acid sequence shown as SEQ ID NO: 3, 4 or 5 or an amino acid sequence which has at least 70% sequence identity thereto may be reduced by at least about 1%, at least about 3%, at least about 5%, at least about 10%, at least about 20%, at least about 30%, at least about 40%, at least about 50%, at least about 60%, at least about 70%, at least about 80%, at least about 90%, at least about 95%, at least about 99% or 100% in relation to a comparable plant which has not been modified to reduce or prevent the expression of a protein comprising the amino acid sequence shown as SEQ ID NO: 3, 4 or 5 or an amino acid sequence which has at least 70% sequence identity thereto. In some embodiments expression or function of a protein comprising the amino acid sequence shown as SEQ ID NO: 3, 4 or 5 or an amino acid sequence which has at least 70% sequence identity thereto may be reduced by between about 5% and about 95%, by between about 10% and about 90%, by between 20% and about 80%, by between 30% and about 70%, or by between about 40% and 60% in relation to a comparable plant which has not been modified to reduce or prevent the expression of a protein comprising the amino acid sequence shown as SEQ ID NO: 3, 4 or 5 or an amino acid sequence which has at least 70% sequence identity thereto.

The terms “to increase the expression or function of a protein” or “increasing expression or function of a protein” are used herein to mean that the amount/level or activity of a protein comprising the amino acid sequence shown as SEQ ID NO: 3, 4 or 5 or an amino acid sequence which has at least 70% sequence identity thereto in the product, method or use of the invention is greater in relation to a comparable product, method or use. For example, a comparable product would be derived from a plant which had not been modified according to the present invention, but in which all other relevant features were the same (e.g. plant species, growing conditions, method of processing, etc).

The expression or function of a protein comprising the amino acid sequence shown as SEQ ID NO: 3, 4 or 5 or an amino acid sequence which has at least 70% sequence identity thereto may be increased in a plant leaf, harvested plant leaf, processed plant leaf, plant product or combinations thereof obtainable or obtained from a plant of the invention when compared with a leaf, harvested plant leaf, processed plant leaf, plant product or combinations thereof obtainable or obtained from a comparable plant which has not been modified to increase the expression of a protein comprising the amino acid sequence shown as SEQ ID NO: 3, 4 or 5 or an amino acid sequence which has at least 70% sequence identity thereto.

“Increased expression” means that a plant is increased in the mRNA level or the protein level in comparison with an expression level of a parent plant of the same breed. The expression level is compared to a corresponding part in the parent plant of the same breed cultured under the same condition. A case where the expression level increases at least 1.1 times greater than that of the parent plant is preferably considered as a case where the expression level is increased. Here, it is more preferable that the expression level of the plant has a significant difference of 5% by a t-test compared with that of the parent plant, in order to be considered that there is an increase in the expression level. It is preferable that the expression levels of the plant and the parent plant be measured at the same time by the same method. However, data stored as background data may be also used.

In one embodiment the expression or function of a protein comprising the amino acid sequence shown as SEQ ID NO: 3, 4 or 5 or an amino acid sequence which has at least 70% sequence identity thereto may be increased by at least about 1%, at least about 3%, at least about 5%, at least about 10%, at least about 20%, at least about 30%, at least about 40%, at least about 50%, at least about 60%, at least about 70%, at least about 80%, at least about 90%, at least about 95%, at least about 99% or 100% in relation to a comparable plant which has not been modified to increase the expression of a protein comprising the amino acid sequence shown as SEQ ID NO: 3, 4 or 5 or an amino acid sequence which has at least 70% sequence identity thereto. In some embodiments the expression or function of a protein comprising the amino acid sequence shown as SEQ ID NO: 3, 4 or 5 or an amino acid sequence which has at least 70% sequence identity thereto may be increased by between about 5% and about 95%, by between about 10% and about 90%, by between 20% and about 80%, by between 30% and about 70%, or by between about 40% and 60% in relation to a comparable plant which has not been modified to increase the expression of a protein comprising the amino acid sequence shown as SEQ ID NO: 3, 4 or 5 or an amino acid sequence which has at least 70% sequence identity thereto.

Any method known in the art for determining the amount/level of a protein comprising the amino acid sequence shown as SEQ ID NO: 3, 4 or 5 or an amino acid sequence which has at least 70% sequence identity thereto may be used in the context of the present invention. For example, known methods such as western blotting, ELISA or in situ hybridization may be used. A modification in the expression of a protein comprising the amino acid sequence shown as SEQ ID NO: 3, 4 or 5 or an amino acid sequence which has at least 70% sequence identity thereto may also be determined by measuring levels of mRNA which encode for said protein. Suitable methods for measuring mRNA are known in the art, for example RT-PCR and RT-qPCR.

Suitably the amount/level or activity of a protein comprising the amino acid sequence shown as SEQ ID NO: 3, 4 or 5 or an amino acid sequence which has at least 70% sequence identity thereto may be modified in a processed leaf.

Suitably the amount/level or activity of a protein comprising the amino acid sequence shown as SEQ ID NO: 3, 4 or 5 or an amino acid sequence which has at least 70% sequence identity thereto may be modified in a plant product.

As used herein the amino acid sequence may comprise, consist essentially of or consist of a sequence shown as SEQ ID NO: 3, 4 or 5 or an amino acid sequence which has at least 70% sequence identity thereto.

In the present Examples, the inventors determined that the amino acid sequence shown as SEQ ID NO: 3 is involved in the control of lateral budding in a tobacco plant.

SEQ ID NO: 3
MATSAAHLIQDQNINIHFDGASLLGKTDTSKASKKGGGGLGGRKALNDIS
NSAKPSALQAKKNYSSNVISIAKDLDATRNKVTDKGGRKALSDLTNSSKL
SAKQAAKNKKLSTAAAANVPTSIAEERFLHDHQKCIKSQRKAMDIDYFLK
EVGLDNDVPVQLAASPRASKLSMKSMLESPVKYFEVEEMPELLMYDKVPR
FGKMETCGDASPSIRSPTSPKLSSMSWWKDGSTPCFTLIESPVLPKH

The present invention encompasses proteins having a degree of sequence identity or sequence homology with the amino acid sequence shown as SEQ ID NO: 3, 4 or 5 (also referred to as a “homologous sequence(s)”). Here, the term “homologue” means an entity having a certain homology with the subject amino acid sequences. Here, the term “homology” can be equated with “identity”.

The homologous amino acid sequence should provide a polypeptide which retains the functional activity the amino acid sequence shown as SEQ ID NO: 3, 4 or 5. In one embodiment the homologous amino acid sequence should provide a polypeptide which retains the functional activity the amino acid sequence shown as SEQ ID NO: 3.

Typically, the homologous sequences will comprise the same active sites and functional domains etc. as the amino acid sequence shown as SEQ ID NO: 3, 4 or 5. In one embodiment, the homologous sequences will comprise the same active sites and functional domains etc. as the amino acid sequence shown as SEQ ID NO: 3. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence which has one or several additions, deletions and/or substitutions compared with amino acid sequence shown as SEQ ID NO: 3, 4 or 5.

In one embodiment the present invention relates to a protein whose amino acid sequence is represented herein as SEQ ID NO: 3, 4 or 5 or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In one embodiment the present invention relates to a nucleic acid sequence (or gene) encoding a protein whose amino acid sequence is represented herein as SEQ ID NO: 3, 4 or 5 or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In one embodiment the present invention relates to a protein whose amino acid sequence is represented herein as SEQ ID NO: 3, 4 or 5 or an amino acid sequence which has at least 70%, at least 75%, at least 80%, at least 85%, at least 90%, at least 95%, at least 97%, or at least 99% sequence identity to SEQ ID NO: 3, 4 or 5.

In one embodiment the present invention relates to a protein whose amino acid sequence is represented herein as SEQ ID NO: 3 or an amino acid sequence which has at least 70%, at least 75%, at least 80%, at least 85%, at least 90%, at least 95%, at least 97%, or at least 99% sequence identity to SEQ ID NO: 3.

In one embodiment the present invention relates to a protein whose amino acid sequence is represented herein as SEQ ID NO: 4 or an amino acid sequence which has at least 70%, at least 75%, at least 80%, at least 85%, at least 90%, at least 95%, at least 97%, or at least 99% sequence identity to SEQ ID NO: 4.

In one embodiment the present invention relates to a protein whose amino acid sequence is represented herein as SEQ ID NO: 5 or an amino acid sequence which has at least 70%, at least 75%, at least 80%, at least 85%, at least 90%, at least 95%, at least 97%, or at least 99% sequence identity to SEQ ID NO: 5.

Nucleic Acid Sequence/Polynucleotide

The present method may comprise providing a mutation in a nucleic acid sequence or polynucleotide which encodes a protein comprising the amino acid sequence shown as SEQ ID NO: 3, 4 or 5 or an amino acid sequence which has at least 70% sequence identity thereto.

The terms “nucleic acid sequence” or “polynucleotide” as used herein refers to an oligonucleotide sequence or polynucleotide sequence, and variant, homologues, fragments and derivatives thereof (such as portions thereof). The nucleotide sequence may be of genomic origin and may be double-stranded or single-stranded whether representing the sense or anti-sense strand.

The terms “nucleic acid sequence” or “polynucleotide” in relation to the present invention may refer to genomic DNA, RNA or cDNA.

In one embodiment, the nucleic acid sequence or polynucleotide which encodes a protein comprising an amino acid sequence shown as SEQ ID NO: 3, 4 or 5 or an amino acid sequence which has at least 70% sequence identity thereto may comprise the nucleic acid sequence shown as SEQ ID NO: 2.

SEQ ID NO: 2
atggctacatcagctgctcacctgattcaggatcagaatatcaatataca
ttttgatggggcttctttgttgggaaagactgatacttccaaggcatcaa
agaaaggaggaggaggtcttggtggaagaaaggcacttaatgacatctca
aactcagcaaagccttctgctctgcaagcaaagaaaaattactcctcgaa
tgtaatttccatcgcaaaagatctcgatgccactcgaaacaaagttactg
acaagggtggcaggaaggcactcagtgatcttacaaactcaagcaagcta
tcagcaaagcaggctgccaagaataagaaactgagtactgctgcagcagc
aaatgttcctacttctatagcggaagagcgatttctgcatgatcatcaga
agtgcatcaaatcacagagaaaagcgatggacattgattattttctaaag
gaagttggactagacaatgatgtccctgtgcagctagcagcatctccacg
tgcatctaaactctcaatgaagtcaatgctagagagccccgtgaagtact
tcgaagtggaagaaatgccagaactgctgatgtatgataaggttcctcga
tttggaaaaatggaaacttgtggagacgcttcgccatctattagatctcc
tacatcaccaaagctatcatctatgagctggtggaaggatggaagcactc
catgtttcacactgattgaatcacccgtcctaccaaagcattga

A genomic DNA sequence which is transcribed into the nucleic acid sequence shown as SEQ ID NO: 2 may comprise the nucleic acid sequence or polynucleotide shown as SEQ ID NO: 1.

SEQ ID NO: 1
ccaaaaaaacgcaaaagaatatttgagaaatcatcaacagatagtttacg
cagttacgaagaagaaaacttgataatcttgtggataaattctctgaaga
aggactgaaaatttctctattctgtgagttttctgtgttcttcaatctac
catcttgcttttgatcgctgagaacattacatgttttcttcaagaatgac
aatttcaatttatctcttctaactgctatgcttctatgttaatatctcaa
cgcttttacatttcctaattggggttgaaattttctagttttttgttatt
tttattatcctgggttcaagttgaagacagatttcgaactcaatttttgc
cgtctactaaagttctaaatttgttgatccacttttccgtttggttaaag
ccttagggttttcggttcttcatactgaattaaagatgtatgattctagg
gttttccgttgttggattaaataataattcttgaaaaaacgaaaagggat
ggcccaacccagagccctcgaggaatttctcaagttttcgaatatttttt
tcatttttaaaggtgaaaagagtttcactgtgttcttctgccttttgtta
aaggagattaacgatttgatcttgatagaaattctaggtacttatggtaa
gataataagcataatcagtttgtttaacttatgcgttaattttggcgcat
cttttcatacctgtgtgcccctgatttgtattcgctaattctgtttcctg
caatcttgctactgctataaatggtcatcttacatggttgcagtgaacat
aaagctatacacatttagtcattctataaaatagtttcagtattatcata
gtacactgacaaaatcgctagagttccacattattaagcttgattcttgt
gtgtagcactcattttttttgttgccaagcttgttctttagggtgtatta
actatctatttttagatgccctcttgaagatgcatttcagatgtagatta
aaacatcttttcttccccataggccccgaatgaactgctttattttgaga
agccatgaccaatgtacttgatctttggcattctgattcttgctacgatt
ataaatgtctaactgaatggttgaagaatataaagctatactcactgagc
aaaaaatgtaacatcacagcacactgagaaaatccccggaatttaaaaca
ttaatgaaactactactatgttgtttataaatctcatttctttgttgttt
tgcttgtactttatgtatgtaatcagttcatttttttcgcttcgatttca
tgttataacgtcaattacatggtgcaggattttattctaattctgctggg
acgcgaatacatggctacatcagctgctcacctgattcaggatcagaata
tcaatatacattttgatggtagaaacctcattagataagcatggagtata
atgctttgatttacaactcgacgttcactaatttttgtctttcatagggg
cttctttgttgggaaagactgatacttccaaggcatcaaagaaaggagga
ggaggtcttggtggaagaaaggcacttaatgacatctcaaactcagcaaa
gccttctgctctgcaagcaaagaaaaattactcctcgaatgtaatttcca
tcgcaaaagatctcgatgccactcgaaacaaagttactgacaagggtggc
aggaaggcactcagtgatcttacaaactcaagcaagctatcagcaaagca
ggctgccaagaataagaaactgagtactgctgcagcagcaaatgttccta
cttctatagcggaagagcgatttctgcatgatcatcagaagtgcatcaaa
tcacagagaaaagcgatggacattgattattttctaaaggaagttggact
agacaatggtaataaaaaaagaaacctgagcaaccatgttttattgtttt
atgaagcagctgataatctgattttcctattgacagatgtccctgtgcag
ctagcagcatctccacgtgcatctaaactctcaatgaagtcaatggtcag
tagcattttgttcctttttatctatttataactcgcttagtcttgtgatt
ctgtaaaataaaagttgtttctaacacctgatgactttgatataccagct
agagagccccgtgaagtacttcgaagtggaagaaatgccagaactgctga
tgtatgataaggttcctcgatttggaaaaatggaaacttgtggagacgct
tcgccatctattagatctcctacatcaccaaagctatcatctatgagctg
gtggaaggatggaagcactccatgtttcacactgattgaatcacccgtcc
taccaaagcattgatgtcttcttctttatgatcacaggcaccctattact
taatggcatcagtcagttggctaaatgactgttgaacttgtttaaattct
ttctggttttggtggcaagaaggcagactagctccttcgtctcgcatgat
ttacattttcggcatattaactgatgcattttggattctatccctcctat
tgtaaaacttaattttctatttcag

As used herein the nucleic acid sequence may comprise, consist essentially of or consist of a sequence shown as SEQ ID NO: 1, 2, 6 or 7 or a nucleic acid sequence (or polynucleotide) which has at least 70% sequence identity thereto.

The present invention also encompasses nucleic acid sequences having a degree of sequence identity or sequence homology with the nucleic acid sequence (or polynucleotide) shown as SEQ ID NO: 1, 2, 6 or 7 (also referred to as a “homologous sequence(s)”). Here, the term “homologue” means an entity having a certain homology with the subject nucleic acid sequences. Here, the term “homology” can be equated with “identity”.

The homologous nucleic acid sequence (or polynucleotide) should encode a polypeptide which retains the functional activity the amino acid sequence shown as SEQ ID NO: 3, 4 or 5. In one embodiment the homologous nucleic acid sequence should encode a polypeptide which retains the functional activity the amino acid sequence shown as SEQ ID NO: 3. Typically, the homologous sequences will encode a protein comprising the same active sites and functional domains etc. as the amino acid sequence shown as SEQ ID NO: 3, 4 or 5. In one embodiment the homologous sequences will encode a protein comprising the same active sites and functional domains etc. as the amino acid sequence shown as SEQ ID NO: 3. Although homology can also be considered in terms of similarity (i.e. amino acid residues having similar chemical properties/functions), in the context of the present invention it is preferred to express homology in terms of sequence identity.

The nucleic acid sequence or polynucleotide may comprise a sequence shown as SEQ ID NO: 1, 2, 6 or 7 or a sequence which has at least 70%, at least 75%, at least 80%, at least 85%, at least 90%, at least 95%, at least 97%, or at least 99% sequence identity to SEQ ID NO: 1, 2, 6 or 7.

The nucleic acid sequence or polynucleotide may comprise a sequence shown as SEQ ID NO: 1 or a sequence which has at least 70%, at least 75%, at least 80%, at least 85%, at least 90%, at least 95%, at least 97%, or at least 99% sequence identity to SEQ ID NO: 1.

The nucleic acid sequence or polynucleotide may comprise a sequence shown as SEQ ID NO: 2 or a sequence which has at least 70%, at least 75%, at least 80%, at least 85%, at least 90%, at least 95%, at least 97%, or at least 99% sequence identity to SEQ ID NO: 2.

The nucleic acid sequence or polynucleotide may comprise a sequence shown as SEQ ID NO: 6 or a sequence which has at least 70%, at least 75%, at least 80%, at least 85%, at least 90%, at least 95%, at least 97%, or at least 99% sequence identity to SEQ ID NO: 6.

The nucleic acid sequence or polynucleotide may comprise a sequence shown as SEQ ID NO: 7 or a sequence which has at least 70%, at least 75%, at least 80%, at least 85%, at least 90%, at least 95%, at least 97%, or at least 99% sequence identity to SEQ ID NO: 7.

Sequence Identity

Homology or identity comparisons can be conducted by eye, or more usually, with the aid of readily available sequence comparison programs. These commercially available computer programs can calculate % homology between two or more sequences.

% homology or % identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

Should Gap Penalties be used when determining sequence identity, the following parameters may be used for pairwise alignment:

| FOR BLAST | | | |
|---|---|---|---|
| GAP OPEN | | 0 | |
| GAP EXTENSION | | 0 | |
| FOR CLUSTAL | DNA | PROTEIN | |
| WORD SIZE | 2 | 1 | K triple |
| GAP PENALTY | 15 | 10 | |
| GAP EXTENSION | 6.66 | 0.1 | |

In one embodiment, BLAST may be used with the gap penalty and gap extension set as defined above.

In one embodiment, CLUSTAL may be used with the gap penalty and gap extension set as defined above.

In some embodiments the gap penalties used for BLAST or CLUSTAL alignment may be different to those detailed above. The skilled person will appreciate that the standard parameters for performing BLAST and CLUSTAL alignments may change periodically and will be able to select appropriate parameters based on the standard parameters detailed for BLAST or CLUSTAL alignment algorithms at the time.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence may be determined over the whole sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also relates to nucleotide sequences that can hybridise to the nucleotide sequences of the present invention (including complementary sequences of those presented herein).

Preferably, hybridisation is determined under stringent conditions (e.g. 50° C. and 0.2×SSC {1×SSC=0.15 M NaCl, 0.015 M Na₃citrate pH 7.0}).

More preferably, hybridisation is determined under high stringent conditions (e.g. 65° C. and 0.1×SSC {1×SSC=0.15 M NaCl, 0.015 M Na₃citrate pH 7.0}).

Reducing or Preventing Expression

Any method known in the art for reducing or preventing the expression or function of a protein may be used in the present method.

By way of example, the present method may comprise:
providing a mutation in a nucleic acid sequence which encodes a protein comprising the amino acid sequence shown as SEQ ID NO: 3, 4 or 5 or an amino acid sequence which has at least 70% sequence identity thereto;
providing a mutation in a regulatory region (e.g. a promoter and an enhancer) which contributes to controlling the expression of a protein comprising the amino acid sequence shown as SEQ ID NO: 3, 4 or 5 or an amino acid sequence which has at least 70% sequence identity thereto;
providing an antisense RNA, siRNA or miRNA which reduces the level of nucleic acid sequence encoding a protein comprising the amino acid sequence shown as SEQ ID NO: 3, 4 or 5 or an amino acid sequence which has at least 70% sequence identity thereto.

Each of the above approaches results in the reduction or prevention of expression or function of a protein comprising the amino acid sequence shown as SEQ ID NO: 3, 4 or 5 or an amino acid sequence which has at least 70% sequence identity thereto.

As used herein, the term “mutation” encompasses a natural genetic variant or an engineered variant. In particular, the term “mutation” refers to a variation in the amino acid sequence compared to the sequence shown as SEQ ID NO: 3, 4 or 5 or an amino acid sequence which has at least 70% sequence identity thereto which reduces the expression or function of the protein.

In a preferred embodiment, each copy of a nucleic acid sequence encoding a protein comprising a sequence shown as SEQ ID NO: 3, 4, 5 or a sequence which has at least 70% sequence identity thereto which is present in the plant is mutated as defined herein (e.g. each genomic copy of a gene encoding said protein in a plant is mutated). For example, each copy of the gene in the allotetraploid genome of N. tabacum may be mutated.

In a preferred embodiment the plant or plant cell according to the present invention is homozygous.

In one embodiment preferably the plant or plant cell according to the present invention expresses only the mutated nucleic acid. In other words, in some embodiments no endogenous (or endogenous and functional) protein is present in the plants according to the present invention. In other words if any endogenous protein is present it is preferably in an inactive and/or truncated form.

In one embodiment the present method may comprise providing a mutation in the sequence shown as SEQ ID NO: 1, 2, 6 or 7 or a nucleic acid sequence which has at least 70% identity thereto.

The mutation may alter the plant genome such that a nucleic acid sequence encoding a protein comprising the amino acid sequence shown as SEQ ID NO: 3, 4 or 5 or an amino acid sequence which has at least 70% sequence identity thereto is completely or partially deleted or otherwise made non-functional.

The mutation may interrupt the nucleic acid sequence which encodes a protein comprising the amino acid sequence shown as SEQ ID NO: 3, 4 or 5 or an amino acid sequence which has at least 70% sequence identity thereto.

The interruption may cause the nucleic acid sequence to not be transcribed and/or translated.

The nucleic acid sequence may be interrupted, for example, by deleting or otherwise modifying the ATG start codon of the nucleic acid sequence such that translation of the protein is reduced or prevented.

The nucleic acid sequence may comprise one or more nucleotide change(s) that reduce or prevent expression of the protein or affect protein trafficking. For example, expression of the protein may be reduced or prevented by introduction of one or more pre-mature stop codons, a frame shift, a splice mutant or a non-tolerated amino acid substitution in the open reading frame.

A premature stop codon refers to a mutation which introduces a stop codon into the open reading frame and prevents translation of the entire amino acid sequence. The premature stop codon may be a TAG (“amber”), TAA (“ochre”), or TGA (“opal” or “umber”) codon.

A frame-shift mutation (also called a framing error or a reading frame shift) is a mutation caused by indels (insertions or deletions) of a number of nucleotides in a nucleic acid sequence that is not divisible by three. Due to the triplet nature of gene expression by codons, the insertion or deletion can change the reading frame, resulting in a completely different translation from the original. A frameshift mutation will often cause the reading of the codons after the mutation to code for different amino acids. The frameshift mutation will commonly result in the introduction of a premature stop codon.

A splice mutant inserts, deletes or changes a number of nucleotides in the specific site at which splicing takes place during the processing of precursor messenger RNA into mature messenger RNA. The deletion of the splicing site results in one or more introns remaining in mature mRNA and may lead to the production of abnormal proteins.

A non-tolerated amino acid substitution refers to a mutation which causes a non-synonymous amino acid substitution in the protein which results in reduced or ablated function of the protein.

Any method known in the art for providing a mutation in a nucleic acid sequence may be used in the present method. For example, homologous recombination may be used, in which a vector is created in which the relevant nucleic acid sequence(s) are mutated and used to transform plants or plant cells. Recombinant plants or plant cells expressing the mutated sequence may then be selected.

In one embodiment the mutation introduces a premature stop codon in a protein comprising an amino acid sequence shown as SEQ ID NO: 3, 4, 5 or a sequence which has at least 70% sequence identity thereto. For example, the mutation may correspond to a G683A mutation in the nucleic acid sequence shown as SEQ ID NO: 2 (which corresponds to a G2303A mutation in SEQ ID NO: 1), which results in the generation of a premature stop codon (TAG). This causes a stop codon to be introduced at position 228 of the amino acid sequence shown as SEQ ID NO: 3. The resulting amino acid sequence is shown as SEQ ID NO: 8, which lacks 19 amino acids form the C-terminus of SEQ ID NO: 3.

In one embodiment the mutation reduces the activity of the protein in relation to a protein shown as SEQ ID NO: 3, 4 or 5 or a sequence which has at least 70% sequence identity thereto.

In one embodiment the mutation does not alter the level or expression but reduces the activity of the protein in relation to a protein shown as SEQ ID NO: 3, 4 or 5 or a sequence which has at least 70% sequence identity thereto.

The nucleic acid sequence may be wholly or partially deleted. The deletion may be continuous, or may comprise a plurality of sections of sequence. The deletion preferably removes a sufficient amount of nucleotide sequence such that the nucleic acid sequence no longer encodes a functional protein. The deletion may, for example, remove at least 50, 60, 70, 80 or 90% of the coding portion of the nucleic acid sequence.

The deletion may be total, in which case 100% of the coding portion of the nucleic acid sequence is absent, when compared to the corresponding genome an comparable unmodified plant.

Methods for deletion of nucleic acid sequences in plants are known in the art. For example, homologous recombination may be used, in which a vector is created in which the relevant nucleic acid sequence(s) are missing and used to transform plants or plant cells. Recombinant plants or plant cells expressing the new portion of sequence may then be selected.

Plant cells transformed with a vector as described above may be grown and maintained in accordance with well-known tissue culturing methods such as by culturing the cells in a suitable culture medium supplied with the necessary growth factors such as amino acids, plant hormones, vitamins, etc.

Modification of the nucleic acid sequence may be performed using targeted mutagenesis methods (also referred to as targeted nucleotide exchange (TNE) or oligo-directed mutagenesis (ODM)). Targeted mutagenesis methods include, without limitation, those employing zinc finger nucleases, TALENs (see WO2011/072246 and WO2010/079430), Cas9-like, Cas9/crRNA/tracrRNA or Cas9/gRNA CRISPR systems (see WO 2014/071006 and WO2014/093622), meganucleases (see WO2007/047859 and WO2009/059195), or targeted mutagenesis methods employing mutagenic oligonucleotides, possibly containing chemically modified nucleotides for enhancing mutagenesis with sequence complementarity to the gene, into plant protoplasts (e.g., KeyBase® or TALENs).

Alternatively, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate plant lines which comprise a gene encoding a protein having a mutation. TILLING uses traditional chemical mutagenesis (e.g. ethyl methanesulfonate (EMS) mutagenesis) followed by high-throughput screening for mutations. Thus, plants, seeds and tissues comprising a gene having the desired mutation may be obtained.

The method may comprise the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such modified plants. Seeds may, for example, be radiated or chemically treated and the plants may be screened for a modified phenotype.

Modified plants may be distinguished from non-modified plants, i.e., wild type plants, by molecular methods, such as the mutation(s) present in the DNA, and by the modified phenotypic characteristics. The modified plants may be homozygous or heterozygous for the mutation.

In one embodiment the method of reducing or preventing the expression of a protein comprising the amino acid sequence shown as SEQ ID NO: 3, 4 or 5 or an amino acid sequence which has at least 70% sequence identity thereto does not comprise treating the plant with a chemical (e.g. an agrochemical).

Increasing Expression

In one aspect the present invention provides a method for increasing lateral budding in a plant by increasing the expression or function of a protein comprising the sequence shown as SEQ ID NO: 3, 4 or 5 or an amino acid sequence which has at least 70% sequence identity thereto.

In one embodiment the present invention provides a method for increasing lateral budding in a plant by increasing the expression of a protein comprising the sequence shown as SEQ ID NO: 3, 4 or 5 or an amino acid sequence which has at least 70% sequence identity thereto.

The increase in expression can be achieved by any means known to the person skilled in the art.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, U.S. Pat. No. 5,565,350; WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 31 end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

In one embodiment the increased expression may be achieved by the use of gene-editing or targeted mutagenesis.

The method may comprise expressing within the plant a polynucleotide (e.g. an exogenous polynucleotide) comprising a nucleic acid sequence encoding a protein comprising the sequence shown as SEQ ID NO: 3, 4 or 5 or an amino acid sequence which has at least 70% sequence identity thereto.

The polynucleotide sequence may comprise the sequence shown as SEQ ID NO: 2, 6 or 7 of a nucleic acid sequence which has at least 70% sequence identity thereto.

The nucleic acid sequence may be operably linked to with a heterologous promoter for directing transcription of said nucleic acid sequence in said plant.

In some embodiments the promoter may be selected from the group consisting of: a constitutive promoter, a tissue-specific promoter, a developmentally-regulated promoter and an inducible promoter.

In one embodiment the promoter may be a constitutive promoter.

A constitutive promoter directs the expression of a gene throughout the various parts of a plant continuously during plant development, although the gene may not be expressed at the same level in all cell types. Examples of known constitutive promoters include those associated with the cauliflower mosaic virus 35S transcript (Odell J T, Nagy F, Chua N H. (1985). Identification of DNA sequences required for activity of the cauliflower mosaic virus 35S promoter. Nature. 313 810-2), the rice actin 1 gene (Zhang W, McElroy D, Wu R. (1991). Analysis of rice Act1 5′ region activity in transgenic rice plants. Plant Cell 3 1155-65) and the maize ubiquitin 1 gene (Cornejo M J, Luth D, Blankenship K M, Anderson O D, Blechl A E. (1993). Activity of a maize ubiquitin promoter in transgenic rice. Plant Molec. Biol. 23 567-81). Constitutive promoters such as the Carnation Etched Ring Virus (CERV) promoter (Hull R, Sadler J, Longstaff M (1986) The sequence of carnation etched ring virus DNA: comparison with cauliflower mosaic virus and retroviruses. EMBO Journal, 5(2):3083-3090).

The constitutive promoter may be selected from a: a carnation etched ring virus (CERV) promoter, a cauliflower mosaic virus (CaMV 35S promoter), a promoter from the rice actin 1 gene or the maize ubiquitin 1 gene.

The promoter may be a tissue specific promoter. In one embodiment the promoter is a lateral meristem specific promoter.

A tissue-specific promoter is one which directs the expression of a gene in one (or a few) parts of a plant, usually throughout the lifetime of those plant parts. The category of tissue-specific promoter commonly also includes promoters whose specificity is not absolute, i.e. they may also direct expression at a lower level in tissues other than the preferred tissue.

An example of a lateral meristem specific promoter is provided by WO 2006/035221.

In another embodiment the promoter may be a developmentally-regulated promoter.

A developmentally-regulated promoter directs a change in the expression of a gene in one or more parts of a plant at a specific time during plant development. The gene may be expressed in that plant part at other times at a different (usually lower) level, and may also be expressed in other plant parts.

In one embodiment the promoter may be an inducible promoter.

An inducible promoter is capable of directing the expression of a gene in response to an inducer. In the absence of the inducer the gene will not be expressed. The inducer may act directly upon the promoter sequence, or may act by counteracting the effect of a repressor molecule. The inducer may be a chemical agent such as a metabolite, a protein, a growth regulator, or a toxic element, a physiological stress such as heat, wounding, or osmotic pressure, or an indirect consequence of the action of a pathogen or pest. A developmentally-regulated promoter might be described as a specific type of inducible promoter responding to an endogenous inducer produced by the plant or to an environmental stimulus at a particular point in the life cycle of the plant. Examples of known inducible promoters include those associated with wound response, such as described by Warner S A, Scott R, Draper J. ((1993) Plant J. 3 191-201), temperature response as disclosed by Benfey & Chua (1989) (Benfey, P. N., and Chua, N-H. ((1989) Science 244 174-181), and chemically induced, as described by Gatz ((1995) Methods in Cell Biol. 50 411-424).

The present invention also provides a construct or vector comprising a nucleic acid sequence encoding a protein comprising the sequence shown as SEQ ID NO: 3, 4 or 5 or an amino acid sequence which has at least 70% sequence identity thereto, as defined herein.

The present invention further provides the use of a nucleic acid sequence encoding a protein comprising the sequence shown as SEQ ID NO: 3, 4 or 5 or an amino acid sequence which has at least 70% sequence identity thereto to increase and/or expedite lateral budding in a plant.

The present invention also provides a chimaeric construct comprising a promoter operably linked to a nucleic acid sequence encoding a protein comprising the sequence shown as SEQ ID NO: 3, 4 or 5 or an amino acid sequence which has at least 70% sequence identity thereto, as defined herein.

A suitable promoter sequence may be constitutive, non-constitutive, tissue-specific, developmentally-regulated or inducible/repressible.

In one embodiment a suitable promoter may be a promoter selected from the group consisting of: the cauliflower mosaic virus 35S promoter, the Carnation Etch Ring Virus (CERV) promoter, the pea plastocyanin promoter, the rubisco promoter, the nopaline synthase promoter, the chlorophyll a/b binding promoter, the high molecular weight glutenin promoter, the α,β-gliadin promoter, the hordein promoter, the patatin promoter, or a senescence-specific promoter.

The construct may be comprised in a vector. Suitably the vector may be a plasmid.

Exogenous polynucleotides may be introduced into plants according to the present invention by means of suitable vector, e.g. plant transformation vectors. A plant transformation vector may comprise an expression cassette comprising 5′-3′ in the direction of transcription, a promoter sequence, a gene of interest (e.g. nucleic acid sequence encoding a protein comprising the sequence shown as SEQ ID NO: 3, 4 or 5 or an amino acid sequence which has at least 70% sequence identity thereto) coding sequence, optionally including introns, and, optionally a 3′ untranslated, terminator sequence including a stop signal for RNA polymerase and a polyadenylation signal for polyadenylase. The promoter sequence may be present in one or more copies, and such copies may be identical or variants of a promoter sequence as described above. The terminator sequence may be obtained from plant, bacterial or viral genes. Suitable terminator sequences are the pea rbcS E9 terminator sequence, the nos terminator sequence derived from the nopaline synthase gene of Agrobacterium tumefaciens and the 35S terminator sequence from cauliflower mosaic virus, for example. A person skilled in the art will be readily aware of other suitable terminator sequences.

The expression cassette may also comprise a gene expression enhancing mechanism to increase the strength of the promoter. An example of such an enhancer element is one derived from a portion of the promoter of the pea plastocyanin gene, and which is the subject of International patent Application No. WO 97/20056. Suitable enhancer elements may be the nos enhancer element derived from the nopaline synthase gene of Agrobacterium tumefaciens and the 35S enhancer element from cauliflower mosaic virus, for example. These regulatory regions may be derived from the same gene as the promoter DNA sequence or may be derived from different genes, for example from a plant of the family Solanaceae. All of the regulatory regions should be capable of operating in cells of the tissue to be transformed.

The promoter DNA sequence may be derived from the same gene as the gene of interest (e.g. the gene the promoter is going to direct, for instance a gene encoding a the modification of a plant to increase the activity or expression of a protein comprising the sequence shown as SEQ ID NO: 3, 4 or 5 or an amino acid sequence which has at least 70% sequence identity thereto) coding sequence used in the present invention or may be derived from a different gene, from for example from a plant of the family Solanaceae.

The expression cassette may be incorporated into a basic plant transformation vector, such as pBIN 19 Plus, pBI 101, or other suitable plant transformation vectors known in the art. In addition to the expression cassette, the plant transformation vector will contain such sequences as are necessary for the transformation process. These may include the Agrobacterium vir genes, one or more T-DNA border sequences, and a selectable marker or other means of identifying transgenic plant cells.

The term “plant transformation vector” means a construct capable of in vivo or in vitro expression. Preferably, the expression vector is incorporated in the genome of the organism. The term “incorporated” preferably covers stable incorporation into the genome.

Techniques for transforming plants are well known within the art and include Agrobacterium-mediated transformation, for example. The basic principle in the construction of genetically modified plants is to insert genetic information in the plant genome so as to obtain a stable maintenance of the inserted genetic material. A review of the general techniques may be found in articles by Potrykus (Annu Rev Plant Physiol Plant Mol Biol [1991] 42:205-225) and Christon (AgroFood-Industry Hi-Tech Mar./Apr. 1994 17-27).

Typically, in Agrobacterium-mediated transformation a binary vector carrying a foreign DNA of interest, is transferred from an appropriate Agrobacterium strain to a target plant by the co-cultivation of the Agrobacterium with explants from the target plant. Transformed plant tissue is then regenerated on selection media, which selection media comprises a selectable marker and plant growth hormones. An alternative is the floral dip method (Clough & Bent, 1998) whereby floral buds of an intact plant are brought into contact with a suspension of the Agrobacterium strain containing the chimeric gene, and following seed set, transformed individuals are germinated and identified by growth on selective media. Direct infection of plant tissues by Agrobacterium is a simple technique which has been widely employed and which is described in Butcher D. N. et al., (1980), Tissue Culture Methods for Plant Pathologists, eds.: D. S. Ingrams and J. P. Helgeson, 203-208.

Further suitable transformation methods include direct gene transfer into protoplasts using polyethylene glycol or electroporation techniques, particle bombardment, micro-injection and the use of silicon carbide fibres for example.

Transforming plants using ballistic transformation, including the silicon carbide whisker technique are taught in Frame B R, Drayton P R, Bagnaall S V, Lewnau C J, Bullock W P, Wilson H M, Dunwell J M, Thompson J A & Wang K (1994). Production of fertile transgenic maize plants by silicon carbide whisker-mediated transformation is taught in The Plant Journal 6: 941-948) and viral transformation techniques is taught in for example Meyer P, Heidmmm I & Niedenhof I (1992). The use of cassava mosaic virus as a vector system for plants is taught in Gene 110: 213-217. Further teachings on plant transformation may be found in EP-A-0449375.

In a further aspect, the present invention relates to a vector system which carries a nucleotide sequence encoding a gene of interest (e.g. a nucleic acid sequence encoding a protein comprising the sequence shown as SEQ ID NO: 3, 4 or 5 or an amino acid sequence which has at least 70% sequence identity thereto) and introducing it into the genome of an organism, such as a plant. The vector system may comprise one vector, but it may comprise two vectors. In the case of two vectors, the vector system is normally referred to as a binary vector system. Binary vector systems are described in further detail in Gynheung Anetal, (1980), Binary Vectors, Plant Molecular Biology Manual A3, 1-19.

One extensively employed system for transformation of plant cells uses the Ti plasmid from Agrobacterium tumefaciens or a Ri plasmid from Agrobacterium rhizogenes Anetal., (1986), Plant Physiol. 81, 301-305 and Butcher D. N. et al., (1980), Tissue Culture Methods for Plant Pathologists, eds.: D. S. Ingrams and J. P. Helgeson, 203-208. After each introduction method of the desired exogenous gene according to the present invention in the plants, the presence and/or insertion of further DNA sequences may be necessary. The use of T-DNA for the transformation of plant cells has been intensively studied and is described in EP-A-120516; Hoekema, in: The Binary Plant Vector System Offset-drukkerij Kanters B. B., Amsterdam, 1985, Chapter V; Fraley, etal., Crit. Rev. Plant Sci., 4:1-46; and Anetal., EMBO J (1985) 4:277-284.

Plant cells transformed with an exogenous gene encoding a protein of interest (e.g. a protein comprising the sequence shown as SEQ ID NO: 3, 4 or 5 or an amino acid sequence which has at least 70% sequence identity thereto) may be grown and maintained in accordance with well-known tissue culturing methods such as by culturing the cells in a suitable culture medium supplied with the necessary growth factors such as amino acids, plant hormones, vitamins, etc.

The term “transgenic plant” in relation to the present invention includes any plant that comprises an exogenous gene encoding a gene of interest, e.g. a protein comprising the sequence shown as SEQ ID NO: 3, 4 or 5 or an amino acid sequence which has at least 70% sequence identity thereto, as described herein. Preferably the exogenous gene is incorporated in the genome of the plant.

The terms “transgenic plant” and “exogenous gene” do not cover native nucleotide coding sequences in their natural environment when they are under the control of their native promoter which is also in its natural environment.

Thus in one embodiment the present invention relates to a method for producing a transgenic plant comprising introducing, into an unmodified plant, an exogenous gene (chimeric construct or vector) encoding a protein comprising the sequence shown as SEQ ID NO: 3, 4 or 5 or an amino acid sequence which has at least 70% sequence identity thereto.

In one embodiment the present invention relates to a method for producing a transgenic plant comprising transforming a plant cell with a construct or vector (e.g. a chimaeric construct) comprising a nucleic acid encoding a protein comprising the sequence shown as SEQ ID NO: 3, 4 or 5 or an amino acid sequence which has at least 70% sequence identity thereto; and regenerating a plant from the transformed plant cell.

Use of an exogenous nucleic acid sequence (construct or vector or chimaeric construct) in accordance with the present invention for increasing or expediting lateral budding in a plant, e.g. by transformation of the plant with the exogenous nucleic acid sequence (construct or vector or chimaeric construct).

In one embodiment the present invention further relates to a host cell comprising an exogenous nucleic acid sequence (construct or vector or chimaeric construct) in accordance with the present invention.

A mutation in the amino acid sequence shown as SEQ ID NO: 3, 4 or 5 or a sequence which has at least 70% sequence identity thereto may increase the activity of the protein in relation to a protein shown as SEQ ID NO: 3, 4 or 5 or a sequence which has at least 70% sequence identity thereto.

A mutation in the amino acid sequence shown as SEQ ID NO: 3, 4 or 5 or a sequence which has at least 70% sequence identity thereto may not alter the level or expression but may increase the activity of the protein in relation to a protein shown as SEQ ID NO: 3, 4 or 5 or a sequence which has at least 70% sequence identity thereto.

Commercially Desirable Traits

The term “commercially desirable traits” will include traits such as yield, quality, abiotic (for instance drought) stress tolerance, herbicide tolerance and/or biotic (for instance insect, bacteria or fungus) stress tolerance.

Plant Breeding

In one embodiment the present invention provides a method of producing a plant having reduced lateral budding, comprising:
a. crossing a donor plant having reduced lateral budding wherein said donor plant comprises a mutation which reduces or prevents the expression or function of a protein comprising the amino acid sequence shown as SEQ ID NO: 3, 4, 5 or an amino acid sequence which has at least 70% identity thereto with a recipient plant that does not have reduced lateral budding and possesses commercially desirable traits;
b. isolating genetic material from a progeny of said donor plant crossed with said recipient plant; and
c. performing molecular marker-assisted selection with a molecular marker comprising:
   i. identifying an introgressed region comprising a mutation which reduces or prevents the expression or function of a protein comprising the amino acid sequence shown as SEQ ID NO: 3, 4, 5 or an amino acid sequence which has at least 70% identity thereto.

In one embodiment the present invention provides a method of producing a plant having increased lateral budding, comprising:
a. crossing a donor plant having increased lateral budding with a recipient plant that does not have increased lateral budding and possesses commercially desirable traits;
b. isolating genetic material from a progeny of said donor plant crossed with said recipient plant; and
c. performing molecular marker-assisted selection with a molecular marker comprising:
   i. identifying an introgressed region comprising a mutation which increases the expression or function of a protein comprising the amino acid sequence shown as SEQ ID NO: 3, 4, 5 or an amino acid sequence which has at least 70% identity thereto.

The molecular marker assisted selection may comprise performing PCR to identify an introgressed nucleic acid sequence comprising a mutation which reduces, prevents or increases the expression or function of a protein comprising the amino acid sequence shown as SEQ ID NO: 3, 4, 5 or an amino acid sequence which has at least 70% identity thereto.

Plant

In one embodiment the plant referred to herein is of the family Solanaceae.

In particular, the plant may be of the subfamily Cestoideae. For example the plant may be a tomato, potato, aubergine, Petunia or tobacco plant.

Examples of tomato and potato amino acid sequences which may be considered homologous to the amino acid sequence shown as SEQ ID NO: 3 have accession numbers XP_004234436 and XP_006353908. These amino acid sequences are shown as SEQ ID NO: 4 (Solanum lycopersicum) and SEQ ID NO: 5 (Solanum tuberosum) respectively.

SEQ ID NO: 4
MAMLQDQNIN IHFDGASLFG KNETSKALKK GGGLGGRKAL
NDISNSAKPS SLQASKKNST SVISIGKDLN ATKNKFIAGT
KDNLAKVPDK GGRKALTDLT NSSKPSAKQG SKKGFDKKWS
AAAAANIPTS IAEEQFLHDH KECIKAQRKV IDMDFFLKEV
GLDNDIPVQP LASPHASKLS MKSMSLTYQL ETPVKKHFEV
DEMPELLMCD QDPQCDKMGT CGGDSSPSLG SPISPKLSYM
SWKDVSDPCF TLTSSPDRQK Y
SEQ ID NO: 5
MAMLQDQNIN IHFDGASLFG KNDTSKALKK GGGLGGRKAL
NDISNSAKPS SLQASKKNSA SVISIGKDLN ATKNKFIAGT
KDNLAKVPDK GGRKALTDLT NSSKPSAKQG SKKGLDKKLS
AAAAANIPTS IAEEQFLHDH KKCIKAQRKV FDMDFFLKEV
GLENDIPVEL LASPRVSKLS MKSMSLTYQL ETPVKKHFEV
EEMPELLMCD QVPKCEKKGT SGDSSPFLGS PISPKLSSMS
WKDVSDPCFT LTGTPDRQKY

SEQ ID NO: 4 has 67% sequence identity to SEQ ID NO: 3. SEQ ID NO: 5 has 68% sequence identity to SEQ ID NO: 3.

Examples of tomato and potato nucleic acid sequences which may be considered homologous to a nucleic acid sequence encoding SEQ ID NO: 2 are the nucleic acid sequences given as accession numbers XM_004234388.2 and XM_006353846.1. The predicted coding sequences derived from these are shown as SEQ ID NO: 6 (Solanum lycopersicum) and SEQ ID NO: 7 (Solanum tuberosum) respectively.

SEQ ID NO: 6
atggctatgc ttcaggatca gaatatcaat atacattttg
atggtgcttc tttgtttgga aagaatgaaa cttccaaagc
cctgaagaaa ggaggaggac ttggcggaag aaaagcactt
aatgacatct ccaactcagc aaagccttct tctctgcagg
catcaaagaa aaactccaca agtgttattt ccatcgggaa
agatcttaat gccaccaaaa acaaatttat tgctgggaca
aaggataacc ttgctaaagt acctgacaag ggtggcagga
aagctctcac tgatcttaca aactcaagca agccatcagc
aaagcagggg tccaagaagg gatttgataa gaaatggagt
gctgctgcag cagcaaatat tccaacttct atagctgaag
aacaatttct gcatgatcat aaggagtgca tcaaagcaca
gaggaaggtg atcgacatgg acttttttct gaaggaagtt
ggactagaca atgatatccc tgtgcagccg ctagcatctc
cacatgcatc taaactctca atgaagtcaa tgtctttgac
gtaccagcta gagacccccg tgaagaagca ctttgaggta
gacgagatgc cagaactgct catgtgtgat caggatcctc
aatgtgataa aatgggaact tgtggaggag actcttcacc
ttctctggga tctcctatat caccaaagct atcatatatg
agctggaaag atgtcagcga tccatgtttc accttgacta
gttcacctga tcgaca
SEQ ID NO: 7
atggctatgc ttcaggatca gaatatcaat atacattttg
atggtgcttc tttgtttgga aagaatgata cttccaaagc
cctgaagaaa ggaggaggac ttggcggaag aaaagcactt
aatgacatct ctaactcagc aaagccttct tctctgcagg
catcaaagaa aaactccgcg agtgttattt ccattgggaa
agatcttaat gccaccaaaa acaaatttat tgctgggaca
aaagataacc ttgctaaagt acctgacaag ggtggtagga
aagctctcac tgatcttaca aactcaagca agccatcagc
aaagcagggg tccaagaagg gacttgataa gaaattgagt
gctgctgcag cagcaaatat tccaacttct atagctgaag
aacaatttct gcatgatcat aagaagtgca tcaaagcaca
gaggaaggtg ttcgacatgg acttttttct gaaggaagtt
ggactagaga atgatatccc tgtggagcta ctagcatctc
cacgcgtgtc taaactctca atgaagtcaa tgtctttgac
gtaccagcta gagacccctg tgaagaagca ctttgaggta
gaggagatgc cagaactgct gatgtgtgat caggttccta
aatgtgaaaa aaagggaact tctggagact cttcaccttt
tctgggatct ccaatatcac caaagctatc atctatgagc
tggaaagatg tcagcgatcc atgtttcacc ttgactggaa
cacccgatcg acaaaagtat tga

SEQ ID NO: 6 has 81% identity to SEQ ID NO: 2. SEQ ID NO: 7 has 80% identity to SEQ ID NO: 2.

Tobacco Plants

In one embodiment, the plant is a tobacco plant.

In one embodiment, the present invention provides methods, uses directed to tobacco plants as well as a tobacco cell, a tobacco plant and a plant propagation material.

In embodiments where the plant is a tobacco plant, the protein comprises a sequence shown as SEQ ID NO: 3, or a sequence which has at least 70% sequence identity thereto.

In a preferred embodiment lateral budding is reduced in a tobacco plant by a method according to the present invention. In particular, in a preferred embodiment the present invention provides a method for reducing lateral budding in a tobacco plant which comprises reducing or preventing the expression or function of a protein comprising the sequence shown as SEQ ID NO: 3 or a sequence which has at least 70% sequence identity thereto.

The term “tobacco plant” as used herein refers to a plant in the genus Nicotiana that is used in the production of tobacco products. Non-limiting examples of suitable tobacco plants include N. tabacum and N. rustica (for example, LA B21, LN KY171, TI 1406, Basma, Galpao, Perique, Beinhart 1000-1, and Petico). It is not intended that the term “tobacco” extends to Nicotiana species that are not useful for the production of tobacco products.

Thus, in one embodiment a tobacco plant does include Nicotiana plumbaginifolia.

The tobacco material can be derived from varieties of Nicotiana tabacum species, commonly known as Burley varieties, flue or bright varieties, dark varieties and oriental/Turkish varieties. In some embodiments, the tobacco material is derived from a Burley, Virginia, flue-cured, air-cured, fire-cured, Oriental, or a dark tobacco plant. The tobacco plant may be selected from Maryland tobacco, rare tobacco, speciality tobacco, expanded tobacco or the like.

The use of tobacco cultivars and elite tobacco cultivars is also contemplated herein. The tobacco plant for use herein may therefore be a tobacco variety or elite tobacco cultivar.

Particularly useful Nicotiana tabacum varieties include Burley type, dark type, flue-cured type, and Oriental type tobaccos.

In some embodiments, the tobacco plant may be, for example, selected from one or more of the following varieties: N. tabacum AA 37-1, N. tabacum B 13P, N. tabacum Xanthi (Mitchell-Mor), N. tabacum KT D#3 Hybrid 107, N. tabacum Bel-W3, N. tabacum 79-615, N. tabacum Samsun Holmes NN, F4 from cross N. tabacum BU21×N. tabacum Hoja Parado, line 97, N. tabacum KTRDC#2 Hybrid 49, N. tabacum KTRDC#4 Hybrid 1 10, N. tabacum Burley 21, N. tabacum PM016, N. tabacum KTRDC#5 KY 160 SI, N. tabacum KTRDC#7 FCA, N. tabacum KTRDC#6 TN 86 SI, N. tabacum PM021, N. tabacum K 149, N. tabacum K 326, N. tabacum K 346, N. tabacum K 358, N. tabacum K 394, N. tabacum K 399, N. tabacum K 730, N. tabacum KY 10, N. tabacum KY 14, N. tabacum KY 160, N. tabacum KY 17, N. tabacum KY 8959, N. tabacum KY 9, N. tabacum KY 907, N. tabacum MD 609, N. tabacum McNair 373, N. tabacum NC 2000, N. tabacum PG 01, N. tabacum PG 04, N. tabacum P01, N. tabacum P02, N. tabacum P03, N. tabacum RG 11, N. tabacum RG 17, N. tabacum RG 8, N. tabacum Speight G-28, N. tabacum TN 86, N. tabacum TN 90, N. tabacum VA 509, N. tabacum AS44, N. tabacum Banket A1, N. tabacum Basma Drama B84/31, N. tabacum Basma I Zichna ZP4/B, N. tabacum Basma Xanthi BX 2A, N. tabacum Batek, N. tabacum Besuki Jember, N. tabacum C104, N. tabacum Coker 319, N. tabacum Coker 347, N. tabacum Criollo Misionero, N. tabacum PM092, N. tabacum Delcrest, N. tabacum Djebel 81, N. tabacum DVH 405, N. tabacum Galpao Comum, N. tabacum HB04P, N. tabacum Hicks Broadleaf, N. tabacum Kabakulak Elassona, N. tabacum PM102, N. tabacum Kutsage E1, N. tabacum KY 14×L8, N. tabacum KY 171, N. tabacum LA BU 21, N. tabacum McNair 944, N. tabacum NC 2326, N. tabacum NC 71, N. tabacum NC 297, N. tabacum NC 3, N. tabacum PVH 03, N. tabacum PVH 09, N. tabacum PVH 19, N. tabacum PVH 21 10, N. tabacum Red Russian, N. tabacum Samsun, N. tabacum Saplak, N. tabacum Simmaba, N. tabacum Talgar 28, N. tabacum PM132, N. tabacum Wislica, N. tabacum Yayaldag, N. tabacum NC 4, N. tabacum TR Madole, N. tabacum Prilep HC-72, N. tabacum Prilep P23, N. tabacum Prilep PB 156/1, N. tabacum Prilep P12-2/1, N. tabacum Yaka JK-48, N. tabacum Yaka JB 125/3, N. tabacum TI-1068, N. tabacum KDH-960, N. tabacum TI-1070, N. tabacum TW136, N. tabacum PM204, N. tabacum PM205, N. tabacum Basma, N. tabacum TKF 4028, N. tabacum L8, N. tabacum TKF 2002, N. tabacum TN90, N. tabacum GR141, N. tabacum Basma xanthi, N. tabacum GR149, N. tabacum GR153, and N. tabacum Petit Havana.

Non-limiting examples of varieties or cultivars are: BD 64, CC 101, CC 200, CC 27, CC 301, CC 400, CC 500, CC 600, CC 700, CC 800, CC 900, Coker 176, Coker 319, Coker 371 Gold, Coker 48, CD 263, DF911, DT 538 LC Galpao tobacco, GL 26H, GL 350, GL 600, GL 737, GL 939, GL 973, HB 04P, HB 04P LC, HB3307PLC, Hybrid 403LC, Hybrid 404LC, Hybrid 501 LC, K 149, K 326, K 346, K 358, K394, K 399, K 730, KDH 959, KT 200, KT204LC, KY10, KY14, KY 160, KY 17, KY 171, KY 907, KY907LC, KTY14×L8 LC, Little Crittenden, McNair 373, McNair 944, msKY 14×L8, Narrow Leaf Madole, Narrow Leaf Madole LC, NBH 98, N-126, N-777LC, N-7371 LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, PD 7302 LC, PD 7309 LC, PD 7312 LC ‘Periq'e’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN 97, TN97LC, TN D94, TN D950, TR (Tom Rosson) Madole, VA 309, VA359, AA 37-1, B 13P, Xanthi (Mitchell-Mor), Bel-W3, 79-615, Samsun Holmes NN, KTRDC number 2 Hybrid 49, Burley 21, KY 8959, KY 9, MD 609, PG 01, PG 04, P01, P02, P03, RG 11, RG 8, VA 509, AS44, Banket A1, Basma Drama B84/31, Basma I Zichna ZP4/B, Basma Xanthi BX 2A, Batek, Besuki Jember, C104, Coker 347, Criollo Misionero, Delcrest, Djebel 81, DVH 405, Galpao Comum, HB04P, Hicks Broadleaf, Kabakulak Elassona, Kutsage E1, LA BU 21, NC 2326, NC 297, PVH 21 10, Red Russian, Samsun, Saplak, Simmaba, Talgar 28, Wislica, Yayaldag, Prilep HC-72, Prilep P23, Prilep PB 156/1, Prilep P12-2/1, Yaka JK-48, Yaka JB 125/3, TI-1068, KDH-960, TI-1070, TW136, Basma, TKF 4028, L8, TKF 2002, GR141, Basma xanthi, GR149, GR153, Petit Havana. Low converter subvarieties of the above, even if not specifically identified herein, are also contemplated.

In one embodiment the tobacco plant is a Burley type tobacco plant, suitably a Burley PH2517.

In one embodiment the plant propagation material may be obtainable from a tobacco plant of the invention.

A “plant propagation material” as used herein refers to any plant matter taken from a plant from which further plants may be produced.

Suitably the plant propagation material may be a seed.

In one embodiment the tobacco cell, tobacco plant and/or plant propagation material may be obtainable (e.g. obtained) by a method according to the invention. In one embodiment the tobacco cell, tobacco plant and/or plant propagation material of the invention may comprise a a mutation in a nucleic acid sequence which encodes a protein comprising the sequence shown as SEQ ID NO: 3 or a sequence which has at least 70% sequence identity thereto.

Suitably a tobacco plant according to the present invention may have reduced lateral budding when compared to an unmodified tobacco plant, wherein the modification is a reduction or prevention of the expression of a protein comprising the sequence shown as SEQ ID NO: 3 or a sequence which has at least 70% sequence identity thereto.

In one embodiment the tobacco plant in accordance with the present invention comprises a tobacco cell of the invention.

In another embodiment the plant propagation material may be obtainable (e.g. obtained) from a tobacco plant of the invention.

In one embodiment there is provided the use of a tobacco cell as provided for in the foregoing embodiments for production of a tobacco product.

Additionally there is provided the use of a tobacco plant as described herein to breed a tobacco plant.

The present invention also provides in another embodiment the use of a tobacco plant of the foregoing embodiments for the production of a tobacco product.

In another embodiment there is provided the use of a tobacco plant of the invention to grow a crop.

Products

The present invention also provides for products obtainable or obtained from tobacco according to the present invention.

In one embodiment there is provided the use of a tobacco plant of the invention to produce a tobacco leaf.

Suitably the tobacco leaf may be subjected to downstream applications such as processing. Thus in one embodiment the use of the foregoing embodiment may provide a processed tobacco leaf. Suitably the tobacco leaf may be subjected to curing, fermenting, pasteurising or combinations thereof.

In another embodiment the tobacco leaf may be cut. In some embodiments the tobacco leaf may be cut before or after being subjected to curing, fermenting, pasteurising or combinations thereof.

In one embodiment the present invention provides a harvested leaf of a tobacco plant of the invention.

In a further embodiment the harvested leaf may be obtainable (e.g. obtained) from a tobacco plant propagated from a propagation material of the present invention.

In another embodiment there is provided a harvest leaf obtainable from a method or use of the present invention.

Suitably the harvested leaf may be a cut harvested leaf.

In some embodiments the harvested leaf may comprise viable tobacco cells. In other embodiments the harvested leaf may be subjected to further processing.

There is also provided a processed tobacco leaf.

The processed tobacco leaf may be obtainable from a tobacco plant of the invention. Suitably the processed tobacco leaf may be obtainable from a tobacco plant obtained in accordance with any of the methods and/or uses of the present invention.

In another embodiment the processed tobacco leaf may be obtainable from a tobacco plant propagated form a tobacco plant propagation material according to the present invention.

The processed tobacco leaf of the present invention may be obtainable by processing a harvested leaf of the invention.

The term “processed tobacco leaf” as used herein refers to a tobacco leaf that has undergone one or more processing steps to which tobacco is subjected to in the art. A “processed tobacco leaf” comprises no or substantially no viable cells.

The term “viable cells” refers to cells which are able to grow and/or are metabolically active. Thus, if a cell is said to not be viable, also referred to as “non-viable” then a cell does not display the characteristics of a viable cell.

The term “substantially no viable cells” means that less than about 5% of the total cells are viable. Preferably, less than about 3%, more preferably less than about 1%, even more preferably less than about 0.1% of the total cells are viable.

In one embodiment the processed tobacco leaf may be processed by one or more of: curing, fermenting and/or pasteurising.

Suitably the processed tobacco leaf may be processed by curing.

Tobacco leaf may be cured by any method known in the art. In one embodiment tobacco leaf may be cured by one or more of the curing methods selected from the group consisting of: air curing, fire curing, flue curing and sun curing.

Suitably the tobacco leaf may be air cured.

Typically air curing is achieved by hanging tobacco leaf in well-ventilated barns and allowing to dry. This is usually carried out over a period of four to eight weeks. Air curing is especially suitable for burley tobacco.

Suitably the tobacco leaf may be fire cured. Fire curing is typically achieved by hanging tobacco leaf in large barns where fires of hardwoods are kept on continuous or intermittent low smoulder and usually takes between three days and ten weeks, depending on the process and the tobacco.

In another embodiment the tobacco leaf may be flue cured. Flue curing may comprise stringing tobacco leaves onto tobacco sticks and hanging them from tier-poles in curing barns. The barns usually have a flue which runs from externally fed fire boxes. Typically this results in tobacco that has been heat-cured without being exposed to smoke. Usually the temperature will be raised slowly over the course of the curing with the whole process taking approximately 1 week.

Suitably the tobacco leaf may be sun cured. This method typically involves exposure of uncovered tobacco to the sun.

Suitably the processed tobacco leaf may be processed by fermenting.

Fermentation can be carried out in any manner known in the art. Typically during fermentation, the tobacco leaves are piled into stacks (a bulk) of cured tobacco covered in e.g. burlap to retain moisture. The combination of the remaining water inside the leaf and the weight of the tobacco generates a natural heat which ripens the tobacco. The temperature in the centre of the bulk is monitored daily. In some methods every week, the entire bulk is opened. The leaves are then removed to be shaken and moistened and the bulk is rotated so that the inside leaves go outside and the bottom leaves are placed on the top of the bulk. This ensures even fermentation throughout the bulk. The additional moisture on the leaves, plus the actual rotation of the leaves themselves, generates heat, releasing the tobacco's natural ammonia and reducing nicotine, while also deepening the colour and improving the tobacco's aroma. Typically the fermentation process continues for up to 6 months, depending on the variety of tobacco, stalk position on the leaf, thickness and intended use of leaf.

Suitably the processed tobacco leaf may be processed by pasteurising. Pasteurising may be particularly preferred when the tobacco leaf will be used to make a smokeless tobacco product, most preferably snus.

Tobacco leaf pasteurisation may be carried out by any method known in the art. For example pasteurisation may be carried out as detailed in J Foulds, L Ramstrom, M Burke, K Fagerstrom. Effect of smokeless tobacco (snus) on smoking and public health in Sweden. Tobacco Control (2003) 12: 349-359, the teaching of which is incorporated herein by reference.

During the production of snus pasteurisation is typically carried out by a process in which the tobacco is heat treated with steam for 24-36 hours (reaching temperatures of approximately 100° C.). This results in an almost sterile product and without wishing to be bound by theory one of the consequences of this is believed to be a limitation of further TSNA formation.

In one embodiment the pasteurisation may be steam pasteurisation.

In some embodiments the processed tobacco leaf may be cut. The processed tobacco leaf may be cut before or after processing. Suitably, the processed tobacco leaf may be cut after processing.

In some embodiments the tobacco plant, harvested leaf of a tobacco plant and/or processed tobacco leaf may be used to extract nicotine. The extraction of nicotine can be achieved using any method known in the art. For example a method for extracting nicotine from tobacco is taught in U.S. Pat. No. 2,162,738 which is incorporated herein by reference.

In another aspect the present invention provides a tobacco product.

In one embodiment the tobacco product may be prepared from a tobacco plant of the invention or a part thereof.

Suitably the tobacco plant or part thereof may be propagated from a tobacco plant propagation material according to the present invention.

The term “part thereof” as used herein in the context of a tobacco plant refers to a portion of the tobacco plant. Preferably the “part thereof” is a leaf of a tobacco plant.

In another embodiment the tobacco product may be prepared from a harvested leaf of the invention.

In a further embodiment the tobacco product may be prepared from a processed tobacco leaf of the invention.

Suitably the tobacco product may be prepared from a tobacco leaf processed by one or more of: curing, fermenting and/or pasteurising.

Suitably the tobacco product may comprise a cut tobacco leaf, optionally processed as per the foregoing embodiment.

In one embodiment the tobacco product may be a smoking article.

As used herein, the term “smoking article” can include smokeable products, such as rolling tobacco, cigarettes, cigars and cigarillos whether based on tobacco, tobacco derivatives, expanded tobacco, reconstituted tobacco or tobacco substitutes.

In another embodiment the tobacco product may be a smokeless tobacco product.

The term “smokeless tobacco product” as used herein refers to a tobacco product that is not intended to be smoked and/or subjected to combustion. In one embodiment a smokeless tobacco product may include snus, snuff, chewing tobacco or the like.

In a further embodiment the tobacco product may be a tobacco heating device.

Typically in heated smoking articles, an aerosol is generated by the transfer of heat from a heat source to a physically separate aerosol-forming substrate or material, which may be located within, around or downstream of the heat source. During smoking, volatile compounds are released from the aerosol-forming substrate by heat transfer from the heat source and entrained in air drawn through the smoking article. As the released compounds cool, they condense to form an aerosol that is inhaled by the user.

Aerosol-generating articles and devices for consuming or smoking tobacco heating devices are known in the art. They can include, for example, electrically heated aerosol-generating devices in which an aerosol is generated by the transfer of heat from one or more electrical heating elements of the aerosol-generating device to the aerosol-forming substrate of a tobacco heating device.

Suitably the tobacco heating device may be an aerosol-generating device.

Preferably the tobacco heating device may be a heat-not-burn device. Heat-not-burn devices are known in the art and release compounds by heating, but not burning, tobacco.

An example of a suitable, heat-not-burn device may be one taught in WO2013/034459 or GB2515502 which are incorporated herein by reference.

In one embodiment the aerosol-forming substrate of a tobacco heating device may be a tobacco product in accordance with the present invention.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

The term “protein”, as used herein, includes proteins, polypeptides, and peptides.

As used herein, the term “amino acid sequence” is synonymous with the term “polypeptide” and/or the term “protein”. In some instances, the term “amino acid sequence” is synonymous with the term “peptide”.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a protein” or “a nucleic acid sequence” includes a plurality of such candidate agents and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Figures and Examples.

### EXAMPLES

### Example 1

### Mutated Nicotiana tabacum Plants with Reduced Lateral Budding

An open-reading frame was identified as a candidate protein involved in lateral budding in Nicotiana tabacum.

Bioinformatics analysis of the candidate open-reading frame identified the genomic sequence (SEQ ID NO: 1), coding-sequence (cds) (SEQ ID NO: 2) and predicted amino acid sequence (SEQ ID NO: 3).

A K326 Nicotiana tabacum mutant with a premature stop codon in the candidate open-reading frame was generated and validated by Sanger sequencing. The mutant comprised a G2303A mutation in the genomic sequence (SEQ ID NO: 1), which resulted in a G683A mutation in the cds (SEQ ID NO: 2) and a premature stop codon at position 228 of the amino acid sequence (SEQ ID NO: 3). This mutant was referred to as TFA1280.

The mature protein resulting from this mutation is shown as SEQ ID NO: 8, which lacks 19 amino acids from the C-terminus of SEQ ID NO: 3.

SEQ ID NO: 8
MATSAAHLIQDQNINIHFDGASLLGKTDTSKASKKGGGGLGGRKALNDIS
NSAKPSALQAKKNYSSNVISIAKDLDATRNKVTDKGGRKALSDLTNSSKL
SAKQAAKNKKLSTAAAANVPTSIAEERFLHDHQKCIKSQRKAMDIDYFLK
EVGLDNDVPVQLAASPRASKLSMKSMLESPVKYFEVEEMPELLMYDKVPR
FGKMETCGDASPSIRSPTSPKLSSMSW*

Digital Phenotyping

TFA1280 homozygous plants and control K326 plants were grown in 3 litre pots with general purpose pot soil. The plants were grown for eleven weeks before being transferred to the belt. At the 8-12^{th} leaf stage plants were topped and leaves pruned. All plants were topped at the same time irrespective of whether or not they had reached flowering. At topping, all but the bottom two or three leaves were removed from the plant.

Post-topping the plants were imaged once a day for 14 days. Daily images were taken using a RGB camera from four side angles at 9, 90, 180 and 270° rotation and one image was taken from the top. Pixel counts were used to determine sucker growth during the experiment. The resulting cleaned detected pixel size dataset was used to fit a growth model from which growth rates (daily increase of pixels) for the different genotypes were estimated. These rates were compared to infer differences between genotypes. The growth model is applied to every plant*angle combination and genotype averages are obtained with correction for relevant factors like greenhouse position where appropriate.

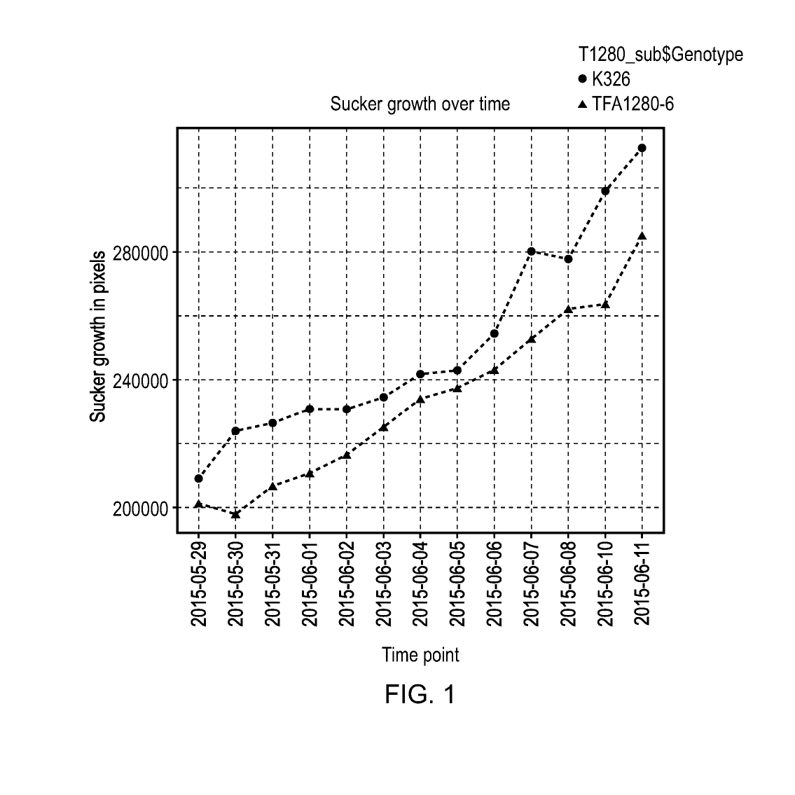

These results demonstrated that the TFA1280 plants had reduced and/or delayed lateral budding (suckering) compared to the control K326 plants (see FIG. 1).

Traditional Biomass Phenotyping

TFA1280 and control K326 plants were grown in the same manner as for the digital phenotyping described above, except that these plants were allowed to reach flowering before being topped and were then topped as required.

Each plant was allowed to continue growing for fourteen days after topping, at which point the top three suckers were removed, pooled, dried and weighed. This weight was used as a measure of the suckering phenotype.

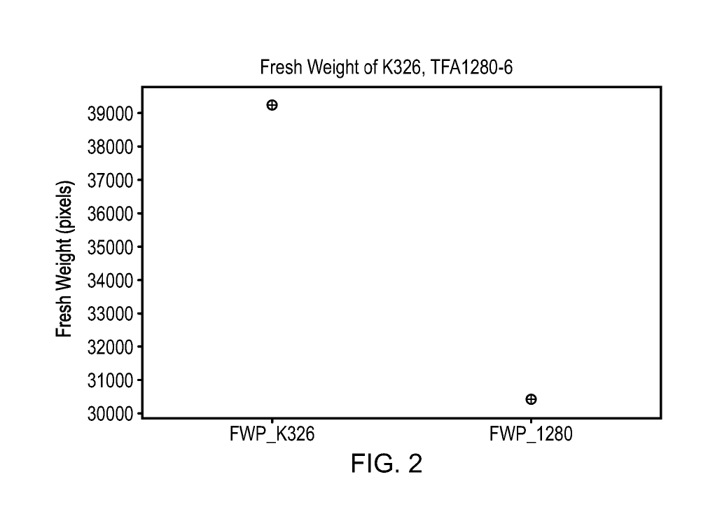

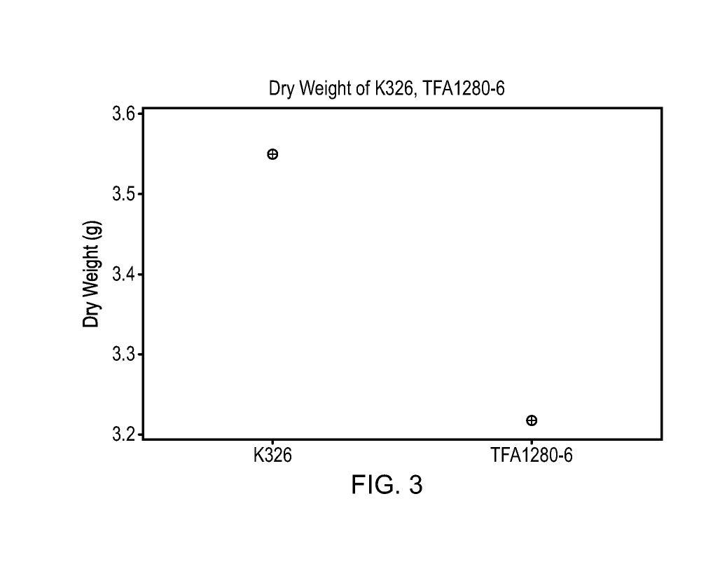

These results demonstrated that the TFA1280 plants had reduced lateral budding (suckering) compared to the control K326 plants (see FIG. 2 & FIG. 3).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for modifying lateral budding in a plant comprising modifying the expression or function of a protein comprising the sequence shown as SEQ ID NO: 3, 4 or 5 or a sequence which has at least 85% sequence identity thereto, wherein the method comprises
i) providing a mutation in a polynucleotide encoding said protein; or
ii) providing a mutation in a regulatory sequence which contributes to controlling the expression of said protein; or
iii) providing an antisense RNA, siRNA or miRNA which reduces the level of nucleic acid sequence encoding said protein; or
iv) increasing expression of the gene encoding said protein or of said protein in comparison with an expression level in a comparable plant.

2. The method according to claim 1 wherein lateral budding is reduced and/or delayed by reducing or preventing the expression or function of said protein.

3. The method according to claim 2 wherein the mutation produces a premature stop codon, a deletion, a splice mutant or codon encoding a non-tolerated amino acid substitution in the polynucleotide encoding said protein.

4. The method according to claim 3 wherein the mutation produces a premature stop codon in the polynucleotide encoding said protein.

5. The method according to claim 4 wherein the mutation produces an amino acid sequence which comprises a premature stop codon at position 228 of SEQ ID NO: 3.

6. A method of producing a plant having reduced and/or delayed lateral budding, comprising:
a. crossing a donor plant having reduced lateral budding wherein the donor plant comprises a mutation which reduces or prevents the expression or function of a protein comprising the amino acid sequence shown as SEQ ID NO: 3, 4, 5 or an amino acid sequence which has at least 85% identity thereto with a recipient plant that does not have reduced lateral budding and possesses commercially desirable traits;
b. isolating genetic material from a progeny of said donor plant crossed with said recipient plant; and
c. performing molecular marker-assisted selection with a molecular marker comprising:
i. identifying an introgressed region comprising a mutation in a polynucleotide encoding a protein as defined in a.

7. The method according to claim 1 wherein the protein comprises the sequence shown as SEQ ID NO: 3, 4, 5 or a sequence which has at least 90%, at least 95%, at least 97%, or at least 99% sequence identity thereto.

8. The method according to claim 1 wherein the protein is encoded by a polynucleotide comprising the sequence shown as SEQ ID NO: 2, 6 or 7 or a sequence which has at least 70% sequence identity thereto.

9. The method according to claim 8 wherein the protein is encoded by a polynucleotide comprising the sequence shown as SEQ ID NO: 2, 6, 7 or a sequence which has at least 75%, at least 80%, at least 85%, at least 90%, at least 95%, at least 97%, or at least 99% sequence identity thereto.

10. (canceled)

11. A plant, plant cell, plant propagation material or harvested leaf comprising:
i) a modified polynucleotide as defined in any of claim 1, part (i) to claim 9; or
ii) an antisense RNA, siRNA or miRNA which reduces the level of nucleic acid sequence encoding the protein as defined in any of claim 1 to claim 9;
iii) or a construct or vector comprising a nucleic acid sequence encoding the protein as defined in any of claim 1 to claim 9.

12.-16. (canceled)

17. The plant, plant cell, plant propagation material or harvested leaf of claim 11, comprising a harvested processed leaf.

18.-19. (canceled)

20. The plant, plant cell, plant propagation material or harvested leaf of claim 11, wherein the plant, plant cell, plant propagation material or harvested leaf is of the family Solanaceae.

21. The cell, plant, plant propagation material, or harvested leaf according to claim 20 wherein the plant is of the subfamily Cestoideae.

22. The cell, plant, plant propagation material, or harvested leaf according to claim 21 wherein the plant is selected from the group consisting of a plant of the genus Nicotiana, a plant of Nicotiana tabacum and a plant of Nicotiana rustica, and wherein the protein comprises a sequence shown as SEQ ID NO: 3, or a sequence which has at least 85% sequence identity thereto and lateral budding is reduced by reducing or preventing the expression or function of said protein.

23. (canceled)

24. The plant, plant cell, plant propagation material or harvested leaf of claim 11, wherein the plant is selected from the group consisting of tomato, cucumber, eggplant, squash, Petunia, Dianthus, Picea, Pinus, Eucalyptus, Populus, potato, tobacco, cotton, lettuce, melon, pea, canola, soybean, sugar beet, sunflower, wheat, barley, rye, rice, maize, pepper, zucchini, Brussels sprouts, broccoli and cauliflower.

25. A tobacco product:
a. prepared from a tobacco plant according to claim 22 or a part thereof;
b. prepared from a tobacco plant or a part thereof, preferably the leaves harvested from the plant, obtained or obtainable by the method according to claim 1;
c. prepared from a tobacco plant, preferably the leaves, propagated from a plant propagation material according to claim 22;
d. prepared from a harvested tobacco leaf according to claim 22;
e. prepared from a processed tobacco leaf according to any of claims 22;
wherein the product comprises
i) a modified polynucleotide as defined in any of claim 1, part i) to 9; or
ii) a mutation in a regulatory sequence which contributes to controlling the expression of the protein as defined in any of claim 1; or
iii) an antisense RNA, siRNA or miRNA which reduces the level of nucleic acid sequence encoding the protein as defined in any of claim 1; or
iv) a construct or vector comprising a nucleic acid sequence encoding the protein as defined in any of claim 1; or
v) a plant cell according to claim 11.

26.-32. (canceled)

33. The plant, plant cell, plant propagation material or harvested leaf of claim 11, comprising a tobacco plant or seed comprising a truncated version of a protein shown as SEQ ID NO: 3 or a sequence which has at least 90%, at least 95%, at least 97% or at least 99% sequence identity thereto, preferably wherein the polynucleotide encoding the truncated protein encodes a premature stop codon at position 228 of SEQ ID NO: 3, wherein said tobacco plant or plant produced from said seed has reduced and/or delayed lateral budding.

34.-37. (canceled)

38. The method of claim 1, wherein said polynucleotide comprises SEQ ID NO:1 or SEQ ID NO:2 or a polynucleotide which has at least 90%, at least 95%, at least 97% or at least 99% sequence identity thereto, and wherein the polynucleotide comprises a mutation at the position corresponding to respectively G2303A and G683A of SEQ ID NO:1 and SEQ ID NO:2, for reducing or delaying lateral budding.

39. The plant, plant cell, plant propagation material or harvested leaf of claim 11, comprising a tomato plant or seed comprising a truncated version of a protein shown as sequence SEQ ID NO:4 or a sequence which has at least 90%, at least 95%, at least 97% or at least 99% sequence identity thereto, preferably wherein the polynucleotide encoding the truncated protein encodes a premature stop codon, e.g. at the position corresponding to 228 of SEQ ID NO:3, wherein said plant or plant produced from said seed has reduced and/or delayed lateral budding.

40.-43. (canceled)

44. The plant, plant cell, plant propagation material or harvested leaf of claim 11, comprising a potato plant or seed comprising a truncated version of a protein shown as SEQ ID NO:5 or a sequence which has at least 90%, at least 95%, at least 97% or at least 99% sequence identity thereto, preferably wherein the polynucleotide encoding the truncated protein encodes a premature stop codon, e.g. at the position corresponding to 228 of SEQ ID NO:3, wherein said plant or plant produced from said seed has reduced and/or delayed lateral budding.

45.-48. (canceled)


SEQUENCE LISTING (8 sequences)

SEQ ID NO: 1 - DNA, 2575 bp, Nicotiana tabacum
ccaaaaaaacgcaaaagaatatttgagaaatcatcaacagatagtttacgcagttacgaagaagaaaacttgataatcttgtggataaattctctgaagaaggactgaaaatttctctattctgtgagttttctgtgttcttcaatctaccatcttgcttttgatcgctgagaacattacatgttttcttcaagaatgacaatttcaatttatctcttctaactgctatgcttctatgttaatatctcaacgcttttacatttcctaattggggttgaaattttctagttttttgttatttttattatcctgggttcaagttgaagacagatttcgaactcaatttttgccgtctactaaagttctaaatttgttgatccacttttccgtttggttaaagccttagggttttcggttcttcatactgaattaaagatgtatgattctagggttttccgttgttggattaaataataattcttgaaaaaacgaaaagggatggcccaacccagagccctcgaggaatttctcaagttttcgaatatttttttcatttttaaaggtgaaaagagtttcactgtgttcttctgccttttgttaaaggagattaacgatttgatcttgatagaaattctaggtacttatggtaagataataagcataatcagtttgtttaacttatgcgttaattttggcgcatcttttcatacctgtgtgcccctgatttgtattcgctaattctgtttcctgcaatcttgctactgctataaatggtcatcttacatggttgcagtgaacataaagctatacacatttagtcattctataaaatagtttcagtattatcatagtacactgacaaaatcgctagagttccacattattaagcttgattcttgtgtgtagcactcattttttttgttgccaagcttgttctttagggtgtattaactatctatttttagatgccctcttgaagatgcatttcagatgtagattaaaacatcttttcttccccataggccccgaatgaactgctttattttgagaagccatgaccaatgtacttgatctttggcattctgattcttgctacgattataaatgtctaactgaatggttgaagaatataaagctatactcactgagcaaaaaatgtaacatcacagcacactgagaaaatccccggaatttaaaacattaatgaaactactactatgttgtttataaatctcatttctttgttgttttgcttgtactttatgtatgtaatcagttcatttttttcgcttcgatttcatgttataacgtcaattacatggtgcaggattttattctaattctgctgggacgcgaatacatggctacatcagctgctcacctgattcaggatcagaatatcaatatacattttgatggtagaaacctcattagataagcatggagtataatgctttgatttacaactcgacgttcactaatttttgtctttcataggggcttctttgttgggaaagactgatacttccaaggcatcaaagaaaggaggaggaggtcttggtggaagaaaggcacttaatgacatctcaaactcagcaaagccttctgctctgcaagcaaagaaaaattactcctcgaatgtaatttccatcgcaaaagatctcgatgccactcgaaacaaagttactgacaagggtggcaggaaggcactcagtgatcttacaaactcaagcaagctatcagcaaagcaggctgccaagaataagaaactgagtactgctgcagcagcaaatgttcctacttctatagcggaagagcgatttctgcatgatcatcagaagtgcatcaaatcacagagaaaagcgatggacattgattattttctaaaggaagttggactagacaatggtaataaaaaaagaaacctgagcaaccatgttttattgttttatgaagcagctgataatctgattttcctattgacagatgtccctgtgcagctagcagcatctccacgtgcatctaaactctcaatgaagtcaatggtcagtagcattttgttcctttttatctatttataactcgcttagtcttgtgattctgtaaaataaaagttgtttctaacacctgatgactttgatataccagctagagagccccgtgaagtacttcgaagtggaagaaatgccagaactgctgatgtatgataaggttcctcgatttggaaaaatggaaacttgtggagacgcttcgccatctattagatctcctacatcaccaaagctatcatctatgagctggtggaaggatggaagcactccatgtttcacactgattgaatcacccgtcctaccaaagcattgatgtcttcttctttatgatcacaggcaccctattacttaatggcatcagtcagttggctaaatgactgttgaacttgtttaaattctttctggttttggtggcaagaaggcagactagctccttcgtctcgcatgatttacattttcggcatattaactgatgcattttggattctatccctcctattgtaaaacttaattttctatttcag

SEQ ID NO: 2 - DNA, 744 bp, Nicotiana tabacum
atggctacatcagctgctcacctgattcaggatcagaatatcaatatacattttgatggggcttctttgttgggaaagactgatacttccaaggcatcaaagaaaggaggaggaggtcttggtggaagaaaggcacttaatgacatctcaaactcagcaaagccttctgctctgcaagcaaagaaaaattactcctcgaatgtaatttccatcgcaaaagatctcgatgccactcgaaacaaagttactgacaagggtggcaggaaggcactcagtgatcttacaaactcaagcaagctatcagcaaagcaggctgccaagaataagaaactgagtactgctgcagcagcaaatgttcctacttctatagcggaagagcgatttctgcatgatcatcagaagtgcatcaaatcacagagaaaagcgatggacattgattattttctaaaggaagttggactagacaatgatgtccctgtgcagctagcagcatctccacgtgcatctaaactctcaatgaagtcaatgctagagagccccgtgaagtacttcgaagtggaagaaatgccagaactgctgatgtatgataaggttcctcgatttggaaaaatggaaacttgtggagacgcttcgccatctattagatctcctacatcaccaaagctatcatctatgagctggtggaaggatggaagcactccatgtttcacactgattgaatcacccgtcctaccaaagcattga

SEQ ID NO: 3 - protein, 247 aa, Nicotiana tabacum
MATSAAHLIQDQNINIHFDGASLLGKTDTSKASKKGGGGLGGRKALNDISNSAKPSALQAKKNYSSNVISIAKDLDATRNKVTDKGGRKALSDLTNSSKLSAKQAAKNKKLSTAAAANVPTSIAEERFLHDHQKCIKSQRKAMDIDYFLKEVGLDNDVPVQLAASPRASKLSMKSMLESPVKYFEVEEMPELLMYDKVPRFGKMETCGDASPSIRSPTSPKLSSMSWWKDGSTPCFTLIESPVLPKH

SEQ ID NO: 4 - protein, 261 aa, Solanum lycopersicum
MAMLQDQNINIHFDGASLFGKNETSKALKKGGGLGGRKALNDISNSAKPSSLQASKKNSTSVISIGKDLNATKNKFIAGTKDNLAKVPDKGGRKALTDLTNSSKPSAKQGSKKGFDKKWSAAAAANIPTSIAEEQFLHDHKECIKAQRKVIDMDFFLKEVGLDNDIPVQPLASPHASKLSMKSMSLTYQLETPVKKHFEVDEMPELLMCDQDPQCDKMGTCGGDSSPSLGSPISPKLSYMSWKDVSDPCFTLTSSPDRQKY

SEQ ID NO: 5 - protein, 260 aa, Solanum tuberosum
MAMLQDQNINIHFDGASLFGKNDTSKALKKGGGLGGRKALNDISNSAKPSSLQASKKNSASVISIGKDLNATKNKFIAGTKDNLAKVPDKGGRKALTDLTNSSKPSAKQGSKKGLDKKLSAAAAANIPTSIAEEQFLHDHKKCIKAQRKVFDMDFFLKEVGLENDIPVELLASPRVSKLSMKSMSLTYQLETPVKKHFEVEEMPELLMCDQVPKCEKKGTSGDSSPFLGSPISPKLSSMSWKDVSDPCFTLTGTPDRQKY

SEQ ID NO: 6 - DNA, 776 bp, Solanum lycopersicum
atggctatgcttcaggatcagaatatcaatatacattttgatggtgcttctttgtttggaaagaatgaaacttccaaagccctgaagaaaggaggaggacttggcggaagaaaagcacttaatgacatctccaactcagcaaagccttcttctctgcaggcatcaaagaaaaactccacaagtgttatttccatcgggaaagatcttaatgccaccaaaaacaaatttattgctgggacaaaggataaccttgctaaagtacctgacaagggtggcaggaaagctctcactgatcttacaaactcaagcaagccatcagcaaagcaggggtccaagaagggatttgataagaaatggagtgctgctgcagcagcaaatattccaacttctatagctgaagaacaatttctgcatgatcataaggagtgcatcaaagcacagaggaaggtgatcgacatggacttttttctgaaggaagttggactagacaatgatatccctgtgcagccgctagcatctccacatgcatctaaactctcaatgaagtcaatgtctttgacgtaccagctagagacccccgtgaagaagcactttgaggtagacgagatgccagaactgctcatgtgtgatcaggatcctcaatgtgataaaatgggaacttgtggaggagactcttcaccttctctgggatctcctatatcaccaaagctatcatatatgagctggaaagatgtcagcgatccatgtttcaccttgactagttcacctgatcgaca

SEQ ID NO: 7 - DNA, 783 bp, Solanum tuberosum
atggctatgcttcaggatcagaatatcaatatacattttgatggtgcttctttgtttggaaagaatgatacttccaaagccctgaagaaaggaggaggacttggcggaagaaaagcacttaatgacatctctaactcagcaaagccttcttctctgcaggcatcaaagaaaaactccgcgagtgttatttccattgggaaagatcttaatgccaccaaaaacaaatttattgctgggacaaaagataaccttgctaaagtacctgacaagggtggtaggaaagctctcactgatcttacaaactcaagcaagccatcagcaaagcaggggtccaagaagggacttgataagaaattgagtgctgctgcagcagcaaatattccaacttctatagctgaagaacaatttctgcatgatcataagaagtgcatcaaagcacagaggaaggtgttcgacatggacttttttctgaaggaagttggactagagaatgatatccctgtggagctactagcatctccacgcgtgtctaaactctcaatgaagtcaatgtctttgacgtaccagctagagacccctgtgaagaagcactttgaggtagaggagatgccagaactgctgatgtgtgatcaggttcctaaatgtgaaaaaaagggaacttctggagactcttcaccttttctgggatctccaatatcaccaaagctatcatctatgagctggaaagatgtcagcgatccatgtttcaccttgactggaacacccgatcgacaaaagtattga

SEQ ID NO: 8 - protein, 227 aa, Artificial Sequence
  mutant TFA1280 mature protein
MATSAAHLIQDQNINIHFDGASLLGKTDTSKASKKGGGGLGGRKALNDISNSAKPSALQAKKNYSSNVISIAKDLDATRNKVTDKGGRKALSDLTNSSKLSAKQAAKNKKLSTAAAANVPTSIAEERFLHDHQKCIKSQRKAMDIDYFLKEVGLDNDVPVQLAASPRASKLSMKSMLESPVKYFEVEEMPELLMYDKVPRFGKMETCGDASPSIRSPTSPKLSSMSW